(19) United States
(12) Patent Application Publication
(10) Pub. No.: US 2023/0081138 A1
(43) Pub. Date: Mar. 16, 2023

(54) DEEP TISSUE OPTICAL SENSING DEVICES AND METHODS

(71) Applicant: Cardiac Pacemakers, Inc., St. Paul, MN (US)

(72) Inventors: Jeffrey E. Stahmann, Ramsey, MN (US); Keith R. Maile, New Brighton, MN (US)

(21) Appl. No.: 17/944,582

(22) Filed: Sep. 14, 2022

Related U.S. Application Data
(60) Provisional application No. 63/244,927, filed on Sep. 16, 2021.

(51) Int. Cl. A61B 5/00

(52) U.S. Cl. CPC A61B 5/0059; A61B 2562/0238

(57) ABSTRACT

Embodiments herein relate to devices and methods for deep tissue optical sensing. In an embodiment, an optical monitoring device is included having a first optical emitter, where the first optical emitter is configured to emit light at a first wavelength. The optical monitoring device includes a first optical detector, where the first optical detector is configured to selectively detect incident light with respect to its angle of incidence on the optical monitoring device. The first optical emitter is configured so that the emitted light from the optical emitter propagates through a tissue at a depth of at least 1 cm into the tissue as measured from a surface of the optical monitoring device. The optical monitoring device is configured to determine a physiological parameter of the tissue using incident light detected by the first optical detector. Other embodiments are also included herein.

This application claims the benefit of U.S. Provisional Application No. 63/244,927, filed Sep. 16, 2021, the content of which is herein incorporated by reference in its entirety.

### FIELD

Embodiments herein relate to devices and methods for deep tissue optical sensing. More specifically, embodiments herein relate to devices and methods for directional optical emission and/or directional optical detection of light to determine a physiological parameter.

### BACKGROUND

Monitoring the overall health and wellness of a patient can include measuring physiological parameters of the patient. In addition, physiological parameters can be monitored in a patient as a part of a comprehensive treatment program to determine the progression of a disease and/or the success of a particular therapy.

Standard measurement techniques used to determine physiological parameters can include those that rely on peripheral monitoring at shallow tissue depths. However, peripheral monitoring at shallow tissue depths may only provide data of questionable accuracy and can be limited by a variety of factors, including low extremity perfusion, vasoconstriction, hypothermia, variation in skin pigmentation, sickle cell anemia, fingernail polish or tattoo ink, motion, ambient light, and the like.

### SUMMARY

In a first aspect, an optical monitoring device is included having a first optical emitter, where the first optical emitter is configured to emit light at a first wavelength. The optical monitoring device can include a first optical detector, where the first optical detector is configured to selectively detect incident light with respect to its angle of incidence on the optical monitoring device. The first optical emitter is configured so that the emitted light from the optical emitter propagates through a tissue at a depth of at least 1 cm into the tissue as measured from a surface of the optical monitoring device. The optical monitoring device is configured to determine a physiological parameter of the tissue using incident light detected by the first optical detector.

In a second aspect, in addition to one or more of the preceding or following aspects, or in the alternative to some aspects, the first optical detector or a portion thereof is angled with respect to a surface of the optical monitoring device.

In a third aspect, in addition to one or more of the preceding or following aspects, or in the alternative to some aspects, the first optical detector includes an angularly selective filter.

In a fourth aspect, in addition to one or more of the preceding or following aspects, or in the alternative to some aspects, the angularly selective filter includes a plurality of angled members, a grating optical filter, an alternating layer filter, a lens, a mirror, or a collimator.

In a fifth aspect, in addition to one or more of the preceding or following aspects, or in the alternative to some aspects, the angularly selective filter is configured to selectively transmit incident light at a first incident angle or a first range of incident angles and selectively block incident light at a second incident angle or second range of incident angles.

In a sixth aspect, in addition to one or more of the preceding or following aspects, or in the alternative to some aspects, the plurality of angled members includes an angle of from 5 degrees to 75 degrees relative to a surface normal of the angularly selective filter.

In a seventh aspect, in addition to one or more of the preceding or following aspects, or in the alternative to some aspects, the first optical detector is configured to detect incident light having a first incident angle of from −75 degrees to +75 degrees relative to the surface normal.

In an eighth aspect, in addition to one or more of the preceding or following aspects, or in the alternative to some aspects, wherein propagation of emitted light through a lung tissue includes propagation of emitted light from 1 cm to 5 cm in depth as measured from a surface of the optical monitoring device.

In a ninth aspect, in addition to one or more of the preceding or following aspects, or in the alternative to some aspects, the first optical emitter and the first optical detector are spaced along a planar surface of the optical monitoring device of from 1 cm to 10 cm apart.

In a tenth aspect, an optical monitoring device is included having a first optical emitter, where the first optical emitter is configured to emit light with a maximum intensity at a predetermined angle from the first optical emitter. The optical monitoring device can include a first optical detector, where the first optical detector is configured to detect incident light. The light from the optical emitter propagates through a tissue and the incident light detected by the first optical detector is used to determine a physiological parameter of the tissue. The light from the optical emitter propagates at a depth of at least 1 cm into the tissue as measured from a surface of the optical monitoring device.

In an eleventh aspect, in addition to one or more of the preceding or following aspects, or in the alternative to some aspects, the first optical emitter is configured to emit light at a first wavelength.

In a twelfth aspect, in addition to one or more of the preceding or following aspects, or in the alternative to some aspects, the first optical emitter and the first optical detector are spaced along a planar surface of the optical monitoring device of from 1 cm to 10 cm apart.

In a thirteenth aspect, in addition to one or more of the preceding or following aspects, or in the alternative to some aspects, the first optical emitter is configured to emit light with a maximum intensity in a direction along a vector that extends at an angle of from 5 degrees to 75 degrees relative to a surface normal the first optical emitter.

In a fourteenth aspect, in addition to one or more of the preceding or following aspects, or in the alternative to some aspects, the first optical emitter is disposed along the optical monitoring device at an angle relative the longitudinal axis of the optical monitoring device.

In a fifteenth aspect, an optical monitoring device is included having a first optical emitter, where the first optical emitter is configured to emit light at a first wavelength. The optical monitoring device includes a first optical detector, where the first optical detector is configured to detect incident light. The optical monitoring device includes a first polarization material disposed on at least a portion of a surface of the first optical emitter and a second polarization material disposed on at least a portion of a surface of the first optical detector. The light from the optical emitter propagates through a tissue, where the incident light detected by the first optical detector is used to determine a physiological parameter of the tissue. The light from the optical emitter propagates at a depth of at least 1 cm into the tissue as measured from a surface of the optical monitoring device.

In a sixteenth aspect, in addition to one or more of the preceding or following aspects, or in the alternative to some aspects, the first polarization material is disposed on an entire surface of the first optical emitter and the second polarization material is disposed on an entire surface first optical detector.

In a seventeenth aspect, in addition to one or more of the preceding or following aspects, or in the alternative to some aspects, a polarization axis of the first polarization material is oriented parallel to a polarization axis of the second polarization material.

In an eighteenth aspect, in addition to one or more of the preceding or following aspects, or in the alternative to some aspects, a polarization axis of the first polarization material is oriented perpendicular to a polarization axis of the second polarization material.

In a nineteenth aspect, in addition to one or more of the preceding or following aspects, or in the alternative to some aspects, a polarization axis of the first polarization material is oriented at an angle in a range of from 0 degrees to 90 degrees to a polarization axis of the second polarization material.

In a twentieth aspect, in addition to one or more of the preceding or following aspects, or in the alternative to some aspects, the first polarization material is disposed about a perimeter of the first optical emitter and the second polarization material is disposed about a perimeter of the first optical detector.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of an implanted optical monitoring device in accordance with the embodiments herein.

FIG. 2 is a schematic view of an implanted optical monitoring device system in accordance with the embodiments herein.

FIG. 3 is a schematic view of a wearable optical monitoring device in accordance with the embodiments herein.

FIG. 4 is a schematic view of a wearable optical monitoring device system in accordance with the embodiments herein.

FIG. 5 is a schematic view of a system including multiple implanted optical monitoring devices in accordance with the embodiments herein.

FIG. 6 is a schematic top view of an optical monitoring device in accordance with various embodiments herein.

FIG. 7 is a schematic top view of an optical monitoring device in accordance with various embodiments herein.

FIG. 8 is a schematic top view of an optical monitoring device in accordance with various embodiments herein.

FIG. 9 is a schematic top view of an optical monitoring device in accordance with various embodiments herein.

FIG. 10 is a schematic cross-sectional view of an optical monitoring device along line 10-10′ of FIG. 9 in accordance with various embodiments herein.

FIG. 11 is a schematic top view of an embodiment of an optical monitoring device in accordance with various embodiments herein.

FIG. 12 is a schematic top view of an embodiment of an optical monitoring device in accordance with various embodiments herein.

FIG. 13 is a schematic cross-sectional view of an optical monitoring device in accordance with various embodiments herein.

FIG. 14 is a schematic top view of an embodiment of an optical monitoring device in accordance with various embodiments herein.

FIG. 15 is a schematic top view of an embodiment of an optical monitoring device in accordance with various embodiments herein.

FIG. 16 is a schematic top view of an embodiment of an optical monitoring device in accordance with various embodiments herein.

FIG. 17 is a schematic cross-sectional view of a subcutaneous implantation site with an optical monitoring device is shown in accordance with various embodiments herein.

FIG. 18 is a representative plot of water absorption of light at various wavelengths illustrating tissue penetration depth in accordance with various embodiments herein.

FIG. 19 is a schematic cross-sectional view of a human thorax with an implanted optical monitoring device positioned therein to measure a physiological parameter in lung tissue in accordance with various embodiments herein.

FIG. 20 is a schematic cross-sectional view of a human thorax with a wearable optical monitoring device positioned thereon to measure a physiological parameter in lung tissue in accordance with various embodiments herein.

FIG. 21 is a cross-sectional view of an embodiment of an optical monitoring device in accordance with various embodiments herein.

FIG. 22 is a cross-sectional view of an embodiment of an optical monitoring device in accordance with various embodiments herein.

FIG. 23 is a schematic diagram of incident light on an optical detector in accordance with various embodiments herein.

FIG. 24 is a schematic cross-sectional view of an embodiment of an optical detector in accordance with various embodiments herein.

FIG. 25 is a schematic cross-sectional view of an embodiment of an optical detector in accordance with various embodiments herein.

FIG. 26 is a schematic cross-sectional view of an embodiment of incident light on an optical detector in accordance with various embodiments herein.

FIG. 27 is a schematic cross-sectional view of an embodiment of an optical monitoring device in accordance with various embodiments herein.

FIG. 28 is a schematic cross-sectional view of an embodiment of an optical monitoring device in accordance with various embodiments herein.

FIG. 29 is a schematic cross-sectional view of an embodiment of an optical monitoring device in accordance with various embodiments herein.

FIG. 30 is a schematic cross-sectional view of an embodiment of an optical monitoring device in accordance with various embodiments herein.

FIG. 31 is a schematic top view of an embodiment of an optical monitoring device in accordance with various embodiments herein.

FIG. 32 is a schematic top view of an embodiment of an optical monitoring device in accordance with various embodiments herein.

FIG. 33 is a schematic diagram of components of an optical monitoring device in accordance with various embodiments herein.

FIG. 34 is a schematic representation oxygenation status versus time in accordance with various embodiments herein.

FIG. 35 is a flow diagram of a method in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As described above, peripheral monitoring of physiological parameters can be limited due to various factors such as sensing limited to shallow tissue depths of under one centimeter (cm). In contrast, the systems, devices, and methods herein are directed to determining and assessing physiological parameters and/or conditions using optical sensing that can penetrate deeply into tissue, such as greater than one centimeter or more into the tissue. In accordance with embodiments herein, various physiological parameters and/or conditions can be determined from the optical properties of the tissue. The physiological parameters and/or conditions can include one or more of an oxygenation status of lung tissue, a pulmonary congestion status, a temperature, and a cardiogenetic airway modulation status, as well as others.

In accordance with embodiments herein, light can be emitted from an optical emitter and can propagate deeply through a patient's tissues and back to an optical detector. As light travels through the tissues of a patient, various optical properties can be affected including transmittance, reflectance, absorption, light attenuation, scattering, wavelength, current, and/or fluorescence. Devices and systems herein can be configured to utilize the optical properties of light having passed through deep tissue to determine physiological parameters and/or conditions. The devices herein can be configured to cause light to penetrate deeply into the tissue by optimizing emitter-detector spacing, wavelength selection, and/or using multiple wavelengths.

As explained in greater detail below, directional light emission and/or directional light detection in accordance with embodiments herein can allow for measurement of physiological parameters and/or detection of conditions at greater depths within the tissue than would otherwise be possible. Directional light emission and/or directional light detection in accordance with embodiments herein can also allow for measurement of physiological parameters and/or detection of conditions at selected depths within the tissue. As such, embodiments herein can include devices and methods for assessing a physiological parameter and/or condition within a deep tissue by utilizing optical monitoring devices that can include one or more of directional optical emitters and/or directional optical detectors. The directional optical emitter can include those that emit light at a predetermined angle or range of angles from the optical emitter and directional optical detectors that detect light having one or more predetermined angles of incidence or range of angles of incidence.

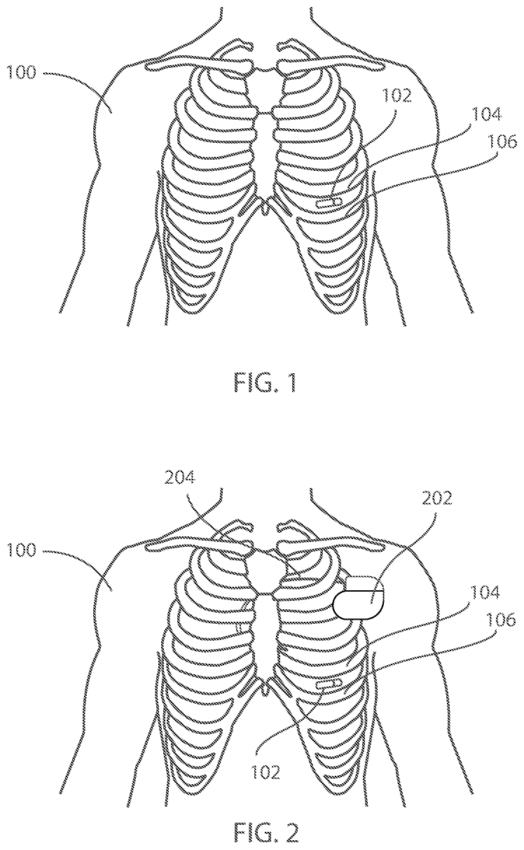

Devices and/or systems herein can take the form of implantable devices, wearable devices, or a combination of both. Specifically, devices and/or systems herein can include optical monitoring devices that are implantable, wearable, or a combination of both. Referring now to FIG. 1, a schematic view of an implantable optical monitoring device is shown in accordance with various embodiments herein. The optical monitoring device 102 is shown in FIG. 1 as being positioned between the left fifth rib 104 and the left sixth rib 106 within the left fifth intercostal space of patient 100. While placement of the optical monitoring device 102 is shown within the left fifth intercostal space, it will be appreciated that the optical monitoring device 102 can be implanted within other locations of the body and, in particular, in or adjacent to other intercostal spaces along either the right side or the left side of a patient's rib cage or at other sites in the body including other anterior, lateral, posterior, superior or inferior thoracic locations.

The implantation depth of the optical monitoring device can be from 1 millimeter (mm) to 25 mm or more below the skin layer of a patient's body. In some embodiments, the implantation depth below the skin layer can be greater than or equal to 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, or 30 mm, or can be an amount falling within a range between any of the foregoing.

In some embodiments, the implantation depth can be tailored to provide an optical monitoring device to lung (or another organ) distance of from 1 mm to 45 mm or more. In some embodiments, the distance between the optical monitoring device and the lung or another organ can be greater than or equal to 1 mm, 2 mm, 3 mm, 4 mm, 5 mm, 6 mm, 7 mm, 8mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm 41 mm, 42 mm, 43 mm, 44 mm, or 45 mm, or can be an amount falling within a range between any of the foregoing.

In some scenarios, the implantable optical monitoring devices embodied herein can be used in combination with various types of implantable therapeutic or monitoring devices, including, but not limited to, implantable cardiac rhythm management devices, implantable pacemakers, implantable cardioverter-defibrillator devices, and the like. The implantable therapeutic devices can be implanted along with the optical monitoring devices described herein. In some embodiments, optical monitoring devices herein (or features and/or components thereof) can be integrated into implantable or wearable therapeutic devices.

Referring now to FIG. 2, a schematic view of an implantable optical monitoring device is shown in accordance with various embodiments herein. The optical monitoring device 102 is shown in FIG. 2 as being positioned between the left fifth rib 104 and the left sixth rib 106 within the left fifth intercostal space of patient 100. FIG. 2 further includes an implantable therapeutic device 202 positioned within the chest of the patient 100. Implantable therapeutic device 202 can be implanted at any position within patient 100 to provide a desired therapy to the patient in conjunction with signals determined by the optical monitoring device, as will be discussed elsewhere herein. In some embodiments, implantable therapeutic device 202 can include one or more electrical stimulation leads 204 placed within the body of patient 100 at or near a treatment site.

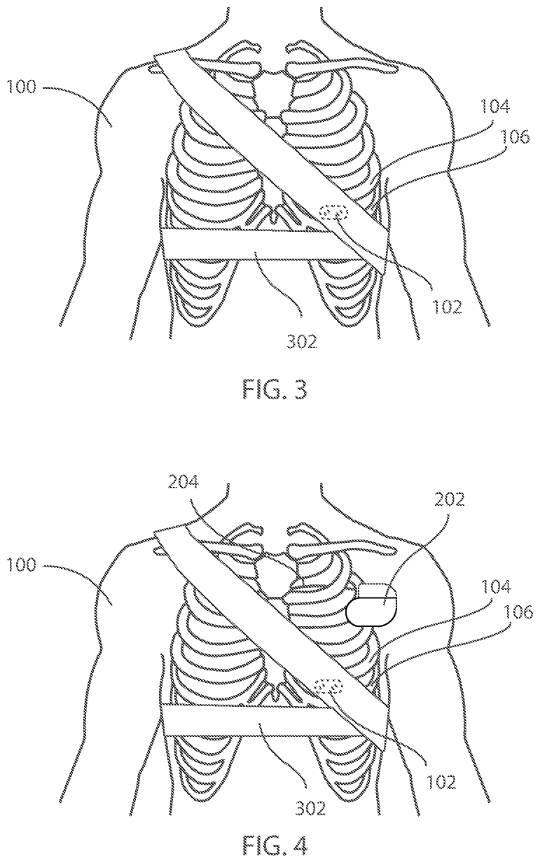

As referenced above, optical monitoring devices herein can include those that are wearable devices. Referring now to FIG. 3, a schematic view of a wearable optical monitoring device is shown in accordance with various embodiments herein. The optical monitoring device 102 can be disposed along a length of a wearable strap 302 and externally positioned between the left fifth rib 104 and the left sixth rib 106 over the left fifth intercostal space of patient 100. While placement of the optical monitoring device 102 is shown placed over the left fifth intercostal space, it will be appreciated that the optical monitoring device 102 can also be positioned over or adjacent to other intercostal spaces along either the right side or the left side of a patient's rib cage. It will be appreciated that the wearable optical monitoring devices herein can be in direct contact with a patient's skin or can be in direct contact with a protective barrier, gel, or film that is placed in direct contact with a patient's skin.

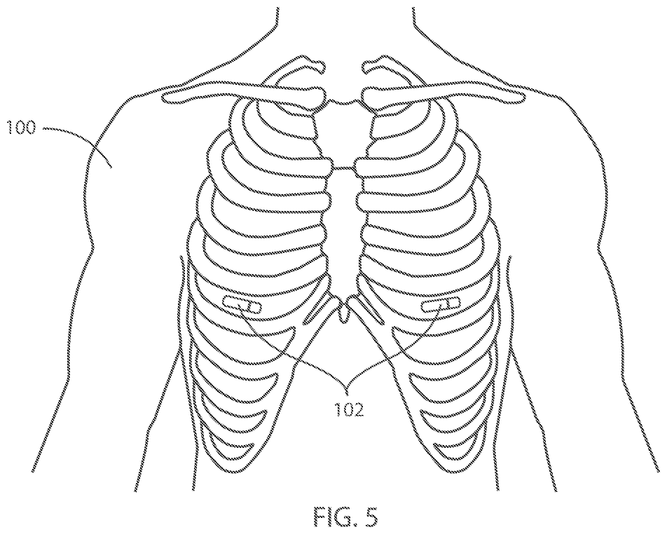

The wearable optical monitoring devices embodied herein can be used in combination with various types of implantable therapeutic devices, such as those described above. The wearable optical monitoring devices embodied herein can be used in combination with various types of implantable monitoring devices, such as implantable cardiac monitors. The implantable therapeutic or implantable monitoring devices can be implanted and used in a system in conjunction with the wearable optical monitoring devices described herein. Referring now to FIG. 4, a schematic view of a wearable optical monitoring device is shown in accordance with various embodiments herein. The wearable optical monitoring device 102 can be disposed along a length of a wearable strap 302 and externally positioned between the left fifth rib 104 and the left sixth rib 106 over the left fifth intercostal space of patient 100 or at another site. FIG. 4 further includes an implantable therapeutic device 202 positioned within the chest of the patient 100. Implantable therapeutic device 202 can be implanted at any position within patient 100 to provide a desired therapy to the patient in conjunction with signals determined by the optical monitoring device, as discussed elsewhere herein. Implantable therapeutic device 202 can include one or more electrical stimulation leads 204 placed within the body of patient 100 at or near a treatment site. In some embodiments, the wearable optical monitoring devices herein can be or included with a patch sensor temporarily affixed to a patient by an adhesive, or it can be retained by a garment worn on the body of a patient.

In some embodiments only a single optical monitoring device may be implanted while in other embodiments multiple optical monitoring devices can be implanted within a patient. Referring now to FIG. 5, a schematic view of a system with multiple implantable optical monitoring devices is shown in accordance with various embodiments herein. The optical monitoring devices 102 are shown in FIG. 5 as being positioned between the fifth rib and the sixth rib within the left fifth intercostal space and right firth intercostal space, respectively. While placement of the optical monitoring devices 102 are shown within the left fifth intercostal space and right fifth intercostal space, it will be appreciated that the optical monitoring devices 102 can be placed within a combination of intercostal spaces along either the right side or the left side of a patient's rib cage or at other sites.

While FIG. 5 shows a combination of two optical monitoring devices implanted within the intercostal spaces of patient 100, it will be appreciated that more than two optical monitoring devices can be implanted within patient 100. In some embodiments, two, three, four, five, or more optical monitoring devices can be implanted within patient 100. In some embodiments one or more of the oxygen monitoring devices is/are implanted and one or more of the oxygen monitoring devices is/are wearable. While not shown in the embodiments as shown in FIGS. 3 and 4, it will be appreciated that a combination of two or more optical monitoring devices can be worn on an exterior of a patient's body as components of the wearable straps 302.

The optical monitoring devices herein can be configured to assess one or more physiological parameters or conditions of a patient's tissue. In various embodiments, the physiological parameter can include one or more of an oxygenation status of lung tissue, a pulmonary congestion status of lung tissue, a temperature, or a cardiogenetic airway modulation status. In various embodiments, the optical monitoring devices herein can be further configured to determine a trend related to magnitude and/or morphology of the physiological parameters or conditions over a time period. In some embodiments, the optical monitoring devices herein can be further configured to determine one or more of a heart rate, a respiratory rate, a tidal volume, or an extravascular lung water concentration, as discussed elsewhere herein. In some embodiments, the optical monitoring devices herein can be further configured to determine respiratory system mechanics based on the physiological parameter.

In various embodiments, optical monitoring devices can be implanted along with at least one secondary sensor. Secondary sensors can include, but are not to be limited to, a pulse oximetry sensor, a chemical sensor, a posture sensor, or a heart rate sensor. The secondary sensors can be used to determine one or more of a heart rate, a respiratory rate, a tidal volume, or an extravascular lung water concentration. In some embodiments, the secondary sensors can be integrated with the optical monitoring device. In other embodiments, the secondary sensors can be physically separate from the optical monitoring device, but in communication therewith.

It will be appreciated that the placement of the optical monitoring device is not restricted to an intercostal space as depicted in FIGS. 1-5. Rather, the optical monitoring device can be placed anywhere in the patient's body where light can propagate a sufficient depth into the body of a patient to measure a physiological parameter or condition of a tissue at any location of a patient's body. In various embodiments, the tissue can include the lung tissue and/or the airway (e.g., trachea, pharynx, nasal cavity, nostril, mouth). However, other tissues are also specifically contemplated herein.

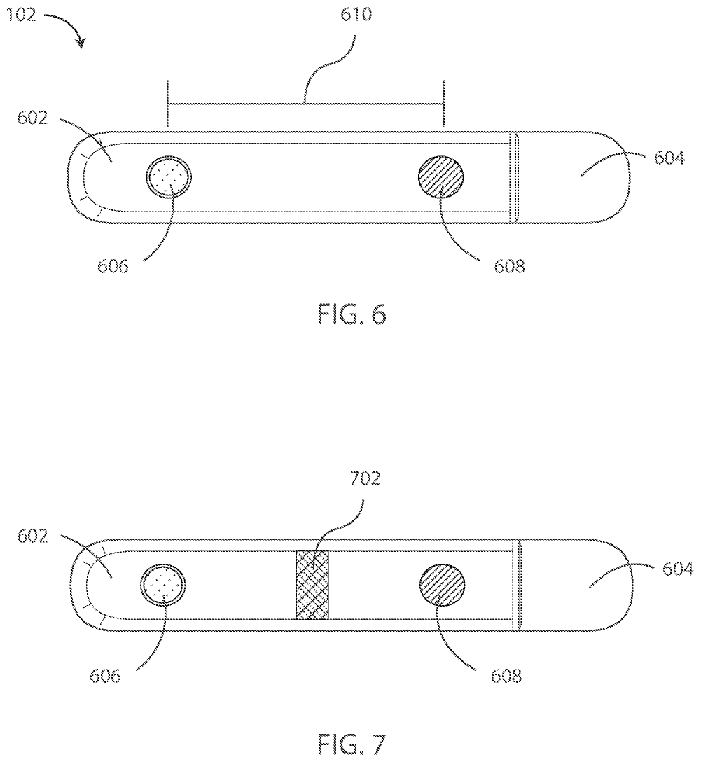

The optical monitoring devices herein can include various components, such as optical emitters, optical detectors, filters, secondary sensors, and optical barriers in various configurations in accordance with the embodiments herein and as shown and described in reference to FIGS. 6-13 and FIGS. 21-31. It will be appreciated that in various embodiments, the optical emitters and optical detectors can be disposed together on the same side of the optical monitoring device, while the secondary sensors can be disposed on the opposite side of the optical monitoring device. In other embodiments, the optical emitters, optical detectors, and secondary sensors can be disposed together on the same side of the optical monitoring device.

Referring now to FIG. 6, an optical monitoring device 102 is shown in accordance with the embodiments herein. In this embodiment, the optical monitoring device 102 can include a housing 602 and a header 604 coupled to the housing 602. Various materials can be used to form the housing 602 and the header 604. In some embodiments, the housing 602 can be formed of a material such as a metal, ceramic, a polymer, or a composite. In some embodiments, part of all of the housing 602 can be transparent to provide a window for optical components of optical monitoring device 102. In some embodiments, the housing 602 can include a transparent window. The header 604 can be formed of various materials, and in some embodiments the header 604 can be formed of a translucent polymer such as an epoxy material. In some embodiments the header 604 can be hollow. In other embodiments the header 604 can be filled with components and/or structural materials such as epoxy or another material such that it is non-hollow. In yet other embodiments, the optical monitoring device 102 can be devoid of a header 604 or can include a header at either end or both ends of the optical monitoring device.

In various embodiments, the optical monitoring device 102 can include a first optical emitter 606 and a first optical detector 608, each coupled to the housing 602. In various embodiments, the optical detectors herein can be configured to selectively detect incident light with respect to its angle of incidence (or range of angles of incidence) on the optical monitoring device as will be discussed in more detail herein. In some embodiments, the optical emitters can be configured to emit light with a maximum intensity at a predetermined angle from the optical emitters. The first optical emitter 606 can be configured to emit light at a UV, visible, or near-infrared wavelength as will be discussed in more detail herein.

The first optical emitter 606 can be configured to emit light at a first wavelength of from 100 nanometers (nm) to 2000 nm. In various embodiments, the first optical emitter can be configured to emit light at a first wavelength of from 600 nm to 1200 nm. In some embodiments, the first optical emitter can be configured to emit light at a first wavelength of from 800 nm to 1000 nm. In various embodiments, the first optical emitter 606 can be configured to emit light at a first wavelength that can be greater than or equal to 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, 1600 nm, 1700 nm, 1800 nm, 1900 nm, or 2000 nm, or can be any wavelength falling within a range between any of the foregoing.

The first optical detector 608 can be configured to detect incident light originating from one or more optical emitters, such as the first optical emitter 606. The first optical detector 608 can be configured to detect light after it has propagated into the tissue of a patient to a given depth from the first optical emitter 606 and back to the first optical detector 608. For example, the optical monitoring device 102 can be configured to provide for the propagation of the emitted light through a lung tissue such that propagation of the emitted light occurs from about 1 centimeter (cm) in depth to 5 cm in depth as measured from a surface of the optical monitoring device 102 to a target tissue. In some embodiments, the depth or propagation of the emitter light can be greater than or equal to 1 cm, 2 cm, 3 cm, 4 cm, or 5 cm, or can be an amount falling within a range between any of the foregoing. In various embodiments, the configuration of the optical monitoring device 102 can allow for the propagation of the emitted light through a lung tissue when a surface of the optical monitoring device 102 is directed to the interior of a patient's body toward the surface of the lung tissue and/or the airway. The detected incident light can be used to measure a physiological parameter in lung and other tissue within a patient, as will be discussed elsewhere herein.

It will be appreciated that first optical emitter 606 and first optical detector 608 can be positioned at any location along optical monitoring device 102 to achieve optimal propagation of light into the tissue of a patient. The first optical emitter 606 and first optical detector 608 can be spaced along a length of the optical monitoring device 102 at a distance 610 from 1 cm to 10 cm apart. In some embodiments, a distance 610 between the first optical emitter and the first optical detector can be greater than or equal to 1 cm, 2 cm, 3 cm, 4 cm, 5 cm, 6 cm, 7 cm, 8 cm, 9 cm, 10 cm, 11 cm, 12 cm, 13 cm, 14 cm, or 15 cm, or can be an amount falling within a range between any of the foregoing. In various embodiments, the first optical emitter and the first optical detector are spaced along a planar surface of the optical monitoring device of from 1 cm to 10 cm apart. In various embodiments, the first optical emitter and the first optical detector are spaced along a flat, planar surface of the optical monitoring device of from 1 cm to 10 cm apart. Subject to other factors, the greater distance 610 is, the greater the depth of propagation of light through the tissue.

The optical monitoring device 102 can take on various dimensions in the length, width, and thickness directions. In a particular embodiment herein, it can be approximately 5 to 10 cm in length, 1 cm to 1.5 cm wide, and 0.25 cm to 1.0 cm thick. In some embodiments, the length of optical monitoring device 102 can be greater than or equal to 1 cm, 2 cm, 3 cm, 4 cm, 5 cm, 6 cm, 7 cm, 8 cm, 9 cm, 10 cm, 11 cm, 12 cm, 13 cm, 14 cm, or 15 cm, or can be an amount falling within a range between any of the foregoing. In some embodiments, the optical monitoring device 102 can be about 0.25 cm, 0.5 cm, 0.75 cm, 1.0 cm, or 2.0 cm in width. In some embodiments the width can be in a range wherein any of the foregoing widths can serve as the upper or lower bound of the range, provided that the upper bound is greater than the lower bound. In some embodiments, the optical monitoring device 102 can be about 0.25 cm, 0.50 cm, 0.75 cm or 1.0 cm, 1.25 cm, 1.50 cm, 1.75 cm, 2.0 cm, 2.25 cm, 2.50 cm, or 3.0 cm thick, or can be an amount falling within a range between any of the foregoing. In some embodiments the thickness can be in a range wherein any of the foregoing thicknesses can serve as the upper or lower bound of the range, provided that the upper bound is greater than the lower bound.

In various embodiments, the optical monitoring devices herein can include an optical barrier device disposed between the optical emitters and optical detectors to prevent a direct path for light emitted from an optical emitter to an optical detector. Referring now to FIG. 7, an optical monitoring device 102 is shown in accordance with the embodiments herein. The optical monitoring device 102 can include the features of the optical monitoring device 102 of FIG. 6 and can further include an optical barrier device 702 disposed in between the first optical emitter 606 and the first optical detector 608. In some embodiments, the optical barrier device 702 can be mounted on the surface of the optical monitoring device 102. In some embodiments, the optical barrier device 702 can be at least partially embedded within the surface of the optical monitoring device 102. The optical barrier device 702 can include various materials including light blocking materials such as opaque polymers, metals, dyed materials, and the like.

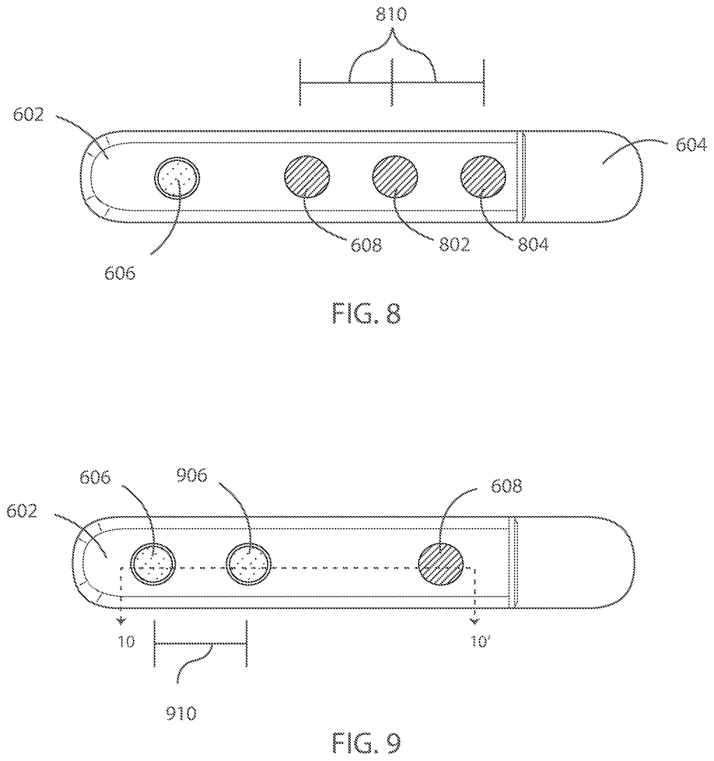

It will be appreciated that the optical monitoring devices embodied herein can include various configurations of multiple optical emitters or optical detectors disposed along a length of the optical monitoring devices. Referring now to FIG. 8, an optical monitoring device 102 is shown in accordance with the embodiments herein. The optical monitoring device 102 can include a housing 602 and a header 604 coupled to the housing 602. The optical monitoring device 102 can include a first optical emitter 606, and an array of optical detectors including a first optical detector 608, a second optical detector 802, and a third optical detector 804, each coupled to the housing 602. The first optical emitter 606 and first optical detector 608 can be spaced along a length of the optical monitoring device 102 as discussed in reference to FIG. 6. The first optical detector 608 and the second optical detector 802, and the second optical detector 802 and the third optical detector 804 can be disposed along a length of the optical monitoring device 102 at a distance 810 of from 1 cm to 5 cm apart. The first optical detector 608, the second optical detector 802, and the third optical detector 804 can be each be configured to detect incident light having a unique incident angle (or range of angles) relative to other optical detectors in the array. In some embodiments, the distance 810 between the first optical detector and the second optical detector can be greater than or equal to 0.25 cm, 0.5 cm, 1 cm, 2 cm, 3 cm, 4 cm, or 5 cm, or more or can be an amount falling within a range between any of the foregoing.

While the device in FIG. 8 shows an array of one by three optical detectors, it will be appreciated that an array can include a number of optical detectors in an array of varying configurations. In various embodiments, an array of optical detectors can include from two, three, four, five, six, seven, eight, nine, ten, or more optical detectors in any configuration on the surface of the optical monitoring device. In various embodiments, each optical detector in the array of optical detectors is configured to detect incident light having a unique incident angle (or range of angles) relative to other optical detectors in the array.

Referring now to FIG. 9, an optical monitoring device 102 is shown in accordance with the embodiments herein. The optical monitoring device 102 can include a housing 602 and a header 604 coupled to the housing 602. The optical monitoring device 102 can include a first optical emitter 606, a second optical emitter 906, and first optical detector 608, each coupled to the housing 602. The first optical emitter 606 and first optical detector 608 can be spaced along a length of the optical monitoring device 102 as discussed in reference to FIG. 6. The first optical emitter 606 and the second optical emitter 906 can be disposed along a length of the optical monitoring device 102 at a distance 910 of from 1 cm to 5 cm or more apart. In some embodiments, the distance 910 between the first optical emitter and the second optical emitter can be greater than or equal to 0.25 cm, 0.5 cm, 1 cm, 2 cm, 3 cm, 4 cm, or 5 cm, or can be an amount falling within a range between any of the foregoing. It will be appreciated, however, that the depth of propagation of light through tissue is dependent on the distance between the emitter and the detector and not the distance between the emitters.

The second optical emitter 906 can be configured to emit light at a second wavelength when present on an optical monitoring device with a first optical emitter. In various embodiments, the second optical emitter 906 can be configured to emit light at a second wavelength different than the first wavelength emitted from the first optical emitter 606. In various embodiments, the second optical emitter 906 can be configured to emit light at a second wavelength of from 100 nm to 2000 nm. In some embodiments, the second optical emitter 906 can be configured to emit light at a second wavelength of from 750 nm to 1500 nm. In various embodiments, the first optical emitter 606 can be configured to emit light at a second wavelength that can be greater than or equal to 700 nm, 800 nm, 900 nm, 1000 nm, 1100 nm, 1200 nm, 1300 nm, 1400 nm, 1500 nm, 1600 nm, 1700 nm, 1800 nm, 1900 nm, or 2000 nm, or can be an amount falling within a range between any of the foregoing. In various embodiments, at least one of the wavelengths of light emitted by the first optical emitter and the second optical emitter can be chosen to be more readily transmitted in air than in bodily fluids.

It will be appreciated that optical emitters herein can be used substantially continuously or only during certain time periods. For example, in some cases, the device and/or system can be configured to measure a physiological parameter continuously or substantially continuously. In such a scenario, an optical emitter can be turned on continuously or intermittently as part of a duty cycle such as a certain fraction of time that the emitter is emitting light. It will be appreciated, however, that continuous operation of an emitter may consume substantial energy and lower the battery life of an implanted device. Thus, as a further example, in some embodiments, the device or system can evaluate a physiological parameter only during certain periods of time. For example, the device or system can evaluate a physiological parameter only when it receives a command to measure a physiological parameter or condition coming from a different device or from a clinician or other system user. As another example, the device or system can evaluate a physiological parameter or condition according to a preset schedule. As another example, the device or system can evaluate a physiological parameter or condition after detecting a particular occurrence or event using one or more sensors, such as an abnormal heart rhythm, an abnormal respiration pattern, high temperature, low oxygenation, or the like. In scenarios where there is more than one emitter, the emitters can be turned on simultaneously or in an alternating pattern.

It will be appreciated that the optical emitters and optical detectors can be formed from various materials. Optical emitters can include a light source such as a light emitting diode (LED), vertical-cavity surface-emitting lasers (VCSELs), electroluminescent (EL) devices, and the like. Optical detectors can include a component selected from the group including of a photodiode, a phototransistor, a charge-coupled device (CCD), a junction field effect transistor (JFET) optical sensor, a complementary metal-oxide semiconductor (CMOS) optical sensor, an integrated photo detector integrated circuit, a light to voltage converter, and the like. Optical emitters and optical detectors are discussed in further detail below.

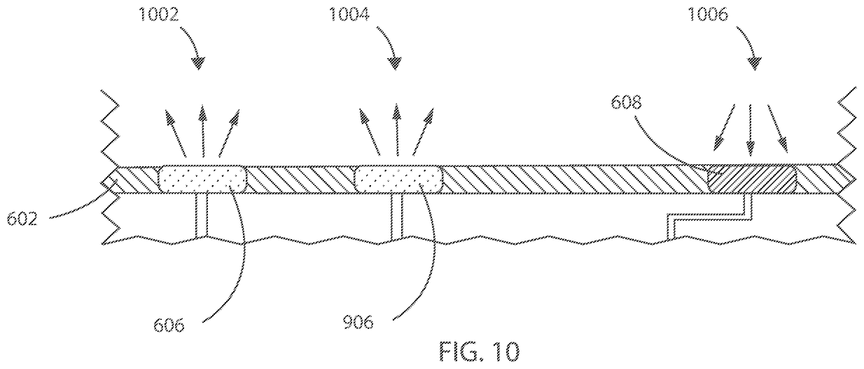

Referring now to FIG. 10, a cross-sectional view of an optical monitoring device is shown in accordance with various embodiments herein. Optical monitoring device 102 includes housing 602 having a first optical emitter 606, a second optical emitter 906, and a first optical detector 608. The first optical emitter 606 can be configured to emit light at a first wavelength 1002 from 100 nm to 2000 nm. The second optical emitter 906 can be configured to emit light at a second wavelength 1004 from 100 nm to 2000 nm. In some embodiments, the first wavelength and second wavelength can be the same. In other embodiments, the first wavelength and the second wavelength can be different. In various embodiments the emitted light of a first wavelength from the first optical emitter can be propagated to a different depth than the emitted light of a second wavelength from the second optical emitter. The first optical detector 608 is configured to detect incident light 1006 that returns to the first optical detector 608 from either the first optical emitter 606, the second optical emitter 906, or both. It will be appreciated that the optical monitoring devices herein can include any combination of one or more optical emitters, optical detectors, filters, and secondary sensors, as described below.

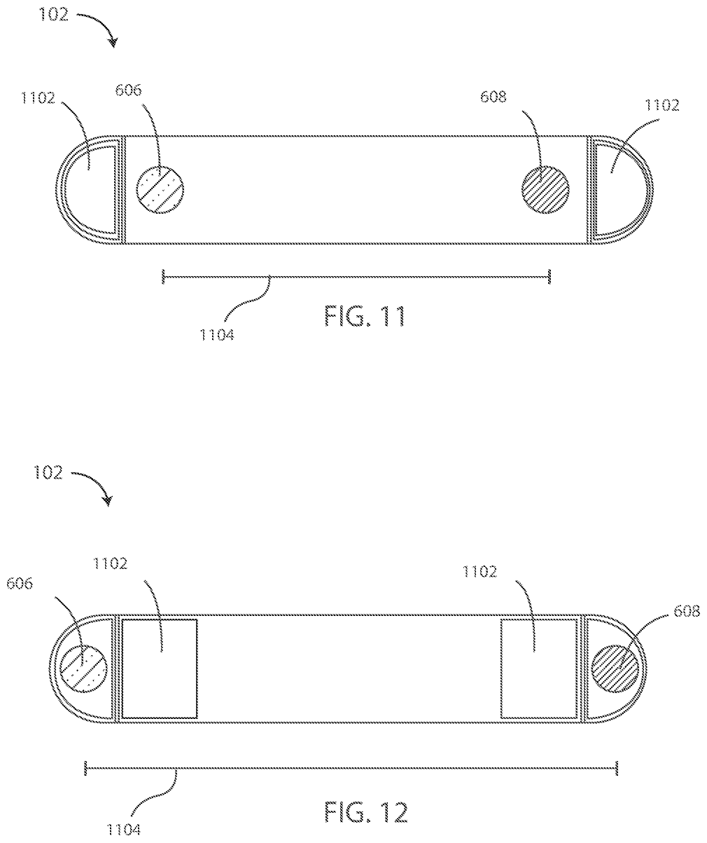

Referring now to FIGS. 11-12, schematic views of additional embodiments of the optical monitoring devices are shown in accordance with various embodiments herein. The optical monitoring devices 102 can include a first optical emitter 606 and a first optical detector 608. While only one optical emitter and one optical detector are shown in the optical monitoring devices 102 of FIGS. 11-12, it will be appreciated that the optical monitoring devices 102 can include more than one optical emitter, more than one optical detector, and a combination of optical emitters and optical detectors. In various embodiments, the optical monitoring devices herein can include an array of optical emitters or an array of optical detectors. The optical monitoring devices 102 can further include one or more electrodes 1102 disposed along a length of the optical monitoring devices 102. Electrodes 1102 can be configured to function as part of a secondary sensor (such as an ECG sensor) or, in some embodiments, deliver an electrical stimulation a therapy to a patient at or near a treatment site, as will be discussed elsewhere herein.

The first optical emitter 606 and the first optical detector 608, as shown in FIGS. 11 and 12 can be disposed along a length of the optical monitoring device separated by a predetermined distance 1104. Predetermined distance 1104 can include a distance such that the first optical emitter 606 and the first optical detector 608 are spaced along a length of the optical monitoring device from 1 cm to 10 cm apart. In some embodiments, a predetermined distance 1104 between the first optical emitter and the first optical detector can be greater than or equal to 1 cm, 2 cm, 3 cm, 4 cm, 5 cm, 6 cm, 7 cm, 8 cm, 9 cm, 10 cm, 11 cm, 12 cm, 13 cm, 14 cm, or 15 cm, or can be an amount falling within a range between any of the foregoing.

In FIG. 11, the first optical emitter 606 and the first optical detector 608 are positioned between electrodes 1102. In some cases, this can be advantageous because depending on the overall dimensions of the device there may be limited space inside of the device in the areas at the ends of the device. However, in FIG. 12, the first optical emitter 606 and the first optical detector 608 are positioned outside of electrodes 1102. In some cases, this can be advantageous to achieve a maximal spacing between the emitter and detector for a given overall device size. This can be important because a significant factor in the depth of light propagation through the tissue is the distance between the emitter and the detector where a greater distance generally results in a greater depth of propagation through the tissue.

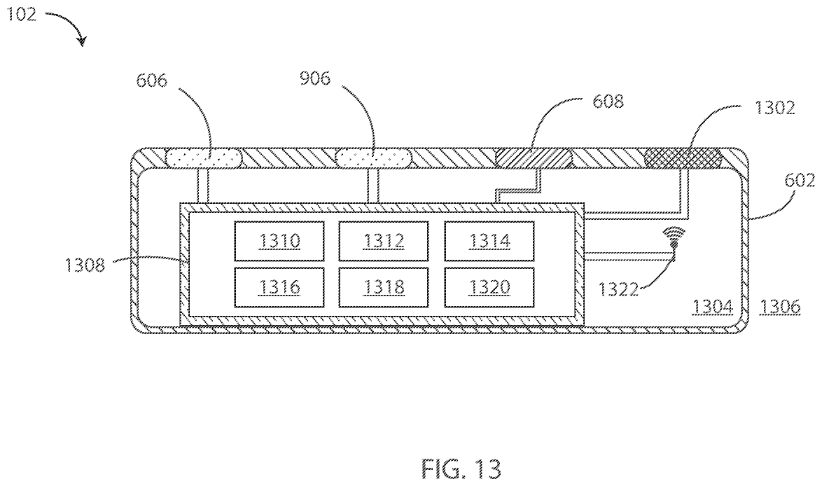

Referring now to FIG. 13, a schematic cross-sectional view of an optical monitoring device 102 is shown in accordance with various embodiments herein. The optical monitoring device 102 can include housing 602. The housing 602 of optical monitoring device 102 can include various materials such as metals, polymers, ceramics, and the like. In some embodiments, all, or part of the housing 602 can be transparent and/or can include transparent portions, such as a transparent window. In some embodiments, the housing 602 can be a single integrated unit. In other embodiments, the housing 602 can include housing 602 and header (not shown in this view), as discussed above. In some embodiments, the housing 602, or one or more portions thereof, can be formed of a biocompatible metal, such as titanium. In some embodiments, one or more segments of the housing 602 can be hermetically sealed.

Housing 602 can define an interior volume 1304 that in some embodiments is hermetically sealed off from the area 1306 outside of optical monitoring device 102. The optical monitoring device 102 can include control circuitry 1308. Control circuitry 1308 can include various components, such as components 1310, 1312, 1314, 1316, 1318, and 1320. In some embodiments, some or all of these components can be integrated and in other embodiments these components can be separate. In some embodiments, the components can include one or more of a microprocessor, memory circuitry (such as random-access memory (RAM) and/or read only memory (ROM)), recorder circuitry, telemetry circuitry, measurement circuitry, chemical sensor interface circuitry, power supply circuitry (which can include one or more batteries), normalization circuitry, optical monitoring device control circuitry, optical emitter control circuitry, optical detector control circuitry, and the like. In some embodiments, recorder circuitry can record the data produced by the optical monitoring device and record time stamps regarding the same. In some embodiments, the circuitry can be hardwired to execute various functions, while in other embodiments the circuitry can be implemented as instructions executing on a microprocessor or other computation device. In various embodiments, optical monitoring device further includes a measurement circuit configured to control operation of the optical emitter(s) and the optical detector(s).

A telemetry interface 1322 can be provided for communicating with external devices such as a programmer, a home-based unit, and/or a mobile unit (e.g., a cellular phone, portable computer, etc.) or a wearable medical device. In some embodiments telemetry interface 1322 can be provided for communicating with implanted devices such as a therapy delivery device (e.g., a pacemaker, cardioverter-defibrillator, or the like) or monitoring-only device (e.g., an implantable loop recorder). In some embodiments, the circuitry can be implemented remotely, via either near-field, far-field, conducted, intra-body or extracorporeal communication, from instructions executing on any of the external or the implanted devices, etc. In some embodiments, the telemetry interface 1322 can be located within housing 602. In some embodiments, the telemetry interface 1322 can be located in a header.

Various optical emitters, optical detectors, and other secondary sensors as described elsewhere herein can be in electrical communication with the circuitry. FIG. 13, shows a first optical emitter 606, a second optical emitter 906, a first optical detector 608 and a secondary sensor 1302 in electrical communication with the control circuitry 1308 within the interior volume 1304. In some embodiments, the control circuitry 1308 is configured to selectively activate first optical emitter 606, the second optical emitter 906, the first optical detector 608 and the secondary sensor 1302. It will be appreciated that the control circuitry 1308 can be configured to selectively activate any number of optical emitters, optical detectors, and secondary sensors.

The secondary sensor 1302 can include, but is not limited to, one or more of a pulse oximetry sensor, a chemical sensor, a posture sensor, or a heart rate sensor. The secondary sensor 1302 can be configured to use electrical, optical, pressure, acoustic, or other techniques. In one example, the secondary sensor 1302 can include one or more electrodes to detect electrical properties including, but not limited, to impedance, electrical potential such as in the case of an ECG signal, and the like. In another example, the secondary sensor 1302 can include a microphone or device to detect pressure waves and/or vibration such as an accelerometer to detect characteristics sounds associated with the heart, lungs, or other physiological activity. In another example, the secondary sensor 1302 can include an accelerometer to detect posture. Aspects of exemplary chemical sensors are described in U.S. Pat. Nos. 7,809,441 and 8,126,554, the content of which is herein incorporated by reference.

It will be appreciated that there can be advantages associated with spacing emitters and detectors as far apart as possible to achieve deep tissue propagation of the emitted light. Generally, a limit on maximum spacing is the overall dimensions of the device and/or portions thereof such as the housing. However, in some embodiments, to achieve greater spacing, another structure can be attached to the device and components such as the emitter and/or detector can be mounted thereon. In this way, a maximum distance of separation can be increased based on the length of the structure added. Such a structure can take various forms. In some embodiments, the structure can be substantially rigid while in other embodiments the structure can be flexible.

While not intending to be bound by theory it can be advantageous to include a flexible structure because a long rigid device can be more likely to cause discomfort for the individual into which the device is implanted (in an implantable embodiment). Thus, in various embodiments, the optical monitoring device can include a flexible member. Flexible members embodied herein can assume many shapes, sizes, and configurations suitable for placement within a patient. In some embodiments, the flexible member can be substantially hollow other than components disposed therein. In some embodiments, the flexible member can be non-hollow and filled with a material such as a polymer, a composite, or the like. In contrast, the housing 602 can be substantially rigid. However, in other embodiments the housing 602 itself can also be flexible.

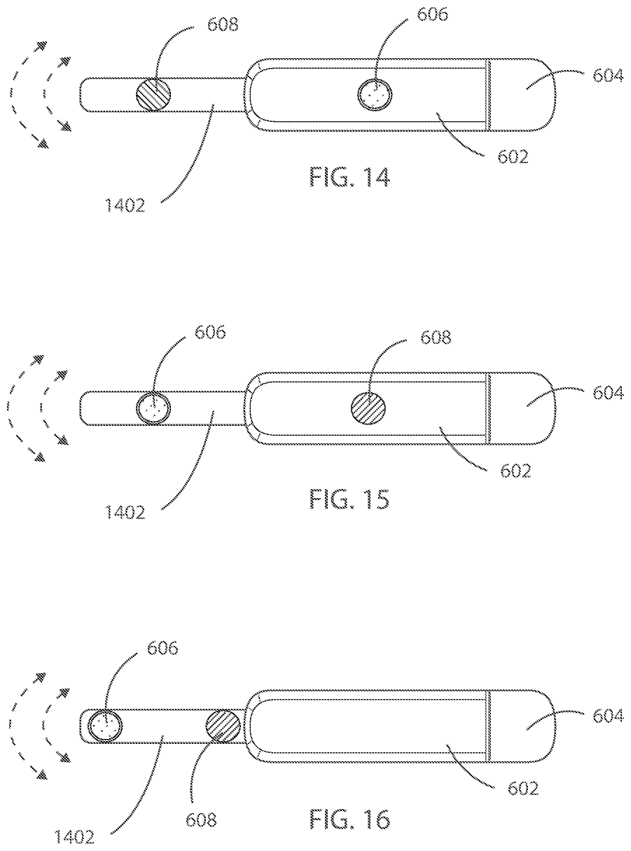

Referring now to FIGS. 14-16 schematic top plan views of optical monitoring devices having flexible members are shown in accordance with various embodiments herein. Specifically referring to FIG. 14, an optical monitoring device 102 is shown including a housing 602 and a header 604 coupled to the housing 602. The optical monitoring device 102 can further include a flexible member 1402 extending form the housing 602. In various embodiments, the flexible member 1402 can be connected to the housing and extend outward in a direction away from the housing 602. As depicted in FIG. 14, the flexible member 1402 can extend outwards from the housing 602 on the opposite side of the housing from the header 604. Alternatively, the flexible member 1402 can extend outwards from the housing 602 on the same side of the housing from the header 604 and/or be an appendage of the header 604 itself. The flexible member 1402 can be constructed form various materials including, for example, a material configured to be bent without damage. In some embodiments, the flexible member 1402 can be formed of a material such as a polymer, an elastomeric polymer, a flexible composite, or the like. In some embodiments, the flexible member 1402 can be of substantially the same diameter and/or circumference as the housing 602, but in other embodiments can be greater or lesser (such as depicted in FIGS. 14-16) in diameter and/or circumference. While not shown in FIGS. 14-16, one or more conductors can interconnect the flexible member and components disposed thereon such as an emitter and/or detector with the other components of the device that may be housed in housing 602.

It will be appreciated that various combinations of optical emitters and optical detectors can be used when a flexible member is present in the optical monitoring devices herein. In various embodiments, the first optical emitter is disposed along a length of the flexible member, while the first optical detector is disposed along the housing. In various embodiments, the first optical detector is disposed along a length of the flexible member, while the first optical emitter is disposed along the housing. In other embodiments, the first optical emitter and the first optical detector are both disposed along a length of the flexible member. However, it is to be understood that various combinations of one or more optical detectors, one or more optical emitters, one or more secondary sensors can be disposed along an optical monitoring device having a flexible member. It will be further understood that in various embodiments, the optical emitters and optical detectors can be disposed together on the same side of the optical monitoring device, while the secondary sensors can be disposed on the opposite side of the optical monitoring device.

The flexible member can take on various dimensions in the length, width, and thickness directions. In a particular embodiment herein, the flexible member can be approximately 1 to 5 cm in length, 0.25 cm to 1.5 cm wide, and 0.25 cm to 1.0 cm thick. In various embodiments, the flexible member is from 1 centimeter (cm) to 3 cm in length. In some embodiments, the length of flexible member 1402 can be greater than or equal to 1 cm, 2 cm, 3 cm, 4 cm, or 5 cm or more, or can be an amount falling within a range between any of the foregoing. In some embodiments, the flexible member 1402 can be about 0.25 cm, 0.5 cm, 0.75 cm, 1.0 cm, or 2.0 cm in width. In some embodiments the length can be in a range wherein any of the foregoing widths can serve as the upper or lower bound of the range, provided that the upper bound is greater than the lower bound. In some embodiments, the flexible member 1402 can be about 0.25 cm, 0.50 cm, 0.75 cm or 1.0 cm, 1.25 cm, 1.50 cm, 1.75 cm, or 2.0 cm thick, or can be an amount falling within a range between any of the foregoing. In some embodiments the thickness can be in a range wherein any of the foregoing thicknesses can serve as the upper or lower bound of the range, provided that the upper bound is greater than the lower bound.

In the embodiment of FIG. 14, the optical monitoring device 102 can include a first optical emitter 606 coupled to the housing and a first optical detector 608 coupled to the flexible member 1402. However, it will be appreciated that the first optical emitter 606 and the first optical detector 608 can be positioned at any location along the optical monitoring device 102, including the flexible member 1402, to achieve optimal propagation of light through the tissue of a patient.

Referring now to FIG. 15, an optical monitoring device 102 is shown in accordance with the embodiments herein. The optical monitoring device 102 can include a housing 602, a header 604 coupled to the housing 602, and a flexible member 1402 extending from the housing 602. In the embodiment of FIG. 15, the optical monitoring device 102 can include a first optical emitter 606 coupled to the flexible member 1402 and a first optical detector 608, coupled to the housing 602.

In some cases, both an emitter and a detector can be disposed on the flexible member. Referring now to FIG. 16, an optical monitoring device 102 is shown in accordance with the embodiments herein. The optical monitoring device 102 can include a housing 602, a header 604 coupled to the housing 602, and a flexible member 1402 extending from the housing 602. The optical monitoring device 102 can include a first optical emitter 606 and a first optical detector 608 each coupled to the flexible member

While only one optical emitter and one optical detector are shown in the optical monitoring devices 102 of FIGS. 14-16, it will be appreciated that the optical monitoring devices 102 can include more than one optical emitter, more than one optical detector, and a combination of optical emitters and optical detectors disposed anywhere across the length of the optical monitoring device.

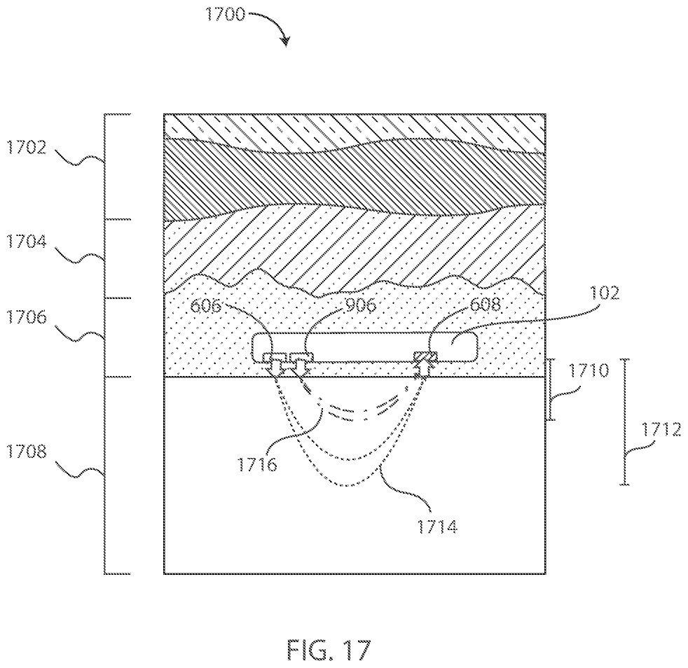

The optical monitoring devices herein can be implanted within various subcutaneous implantation sites or other regions of the inside of the body. Referring now to FIG. 17, a schematic cross-sectional view of a subcutaneous implantation site 1700 with an implanted optical monitoring device 102 is shown in accordance with various embodiments herein. Human skin includes multiple layers including the epidermis 1702 and the dermis 1704. Beneath the layers of human skin and typically above a layer of muscle 1708 is the subcutaneous space that can include a layer of adipose tissue 1706. It will be appreciated that the subcutaneous space further includes additional anatomical structures such as blood vessels, fascia, lymphatic vessels, nervous tissue, hair follicles, and the like.

The optical monitoring device 102 can be disposed at any location within the subcutaneous space. In various embodiments, the optical monitoring device 102 can even be implanted deeper within the body, such as within underlying muscle, within a cavity, such as the chest cavity, adjacent to or within an internal organ of the body, within a blood vessel, or the like. In various embodiments, the optical monitoring device 102 can be held in place via sutures. In some embodiments, the optical monitoring device 102 can include one or more apertures to facilitate its fixation via sutures. The optical monitoring device 102 can be implanted within a patient such that each of the optical emitters and optical detectors included on the optical monitoring device 102 are disposed facing the interior of a patient's body and are directed toward the volume of the tissue to be monitored. In some embodiments, the tissue to be monitored includes the lung tissue, airway tissue cardiovascular tissue, and the like. Implanting the optical monitoring device 102 facing the interior of a patient's body can selectively direct emitted light toward tissues to be illuminated inside the patient's body.

The optical monitoring device 102 shown in FIG. 17 includes a first optical emitter 606, a second optical emitter 906, and a first optical detector 608. In various embodiments, the optical monitoring device 102 can be configured such that the emitted light 1714 from the first optical emitter 606 at a first wavelength is propagated to a first depth 1712 from a surface of the optical monitoring device 102, and the emitted light 1716 from the second optical emitter 906 at a second wavelength is propagated to a second depth 1710 from a surface of the optical monitoring device 102. In various embodiments, the optical monitoring device 102 can be configured such that emitted light 1714 from the first optical emitter 606 is propagated to a different depth than emitted light 1716 from the second optical emitter 906.

In various embodiments, the depth of emitted light propagated into the tissue of the patient can be tailored by changing the distance between the first optical emitter 606 and second optical emitter 906, and the first optical detector 608, where various distances suitable for use between the optical emitters and optical detectors are discussed elsewhere herein. In some embodiments, the depth of emitted light propagated into the tissue of the patient can also be tailored by selecting various wavelengths of light to be emitted by the first optical emitter 606 and second optical emitter 906. In some embodiments, the first wavelength and second wavelength are different wavelengths. Wavelengths suitable for use in the optical monitoring devices described are discussed elsewhere herein.

Without being bound by any particular theory, it is believed that light generally follows a direct path through a medium in the absence of scattering effects. However, in more complex media where scattering is prevalent, it is believed that light propagation through such media occurs by way of random scattering. Biological tissue is complex, as it is comprised of many tissue types with complex molecules and compounds distributed therein. It is believed that light propagation through biological tissue is due to scattering as caused by the difference in the index of refraction of the various molecules and compounds in the tissues. By way of example, scattering of light can occur in the blood due to the difference of the index of refraction between red blood cells and plasma, and can occur in other tissues due to the difference of the index of refraction between cells, including cellular organelles, and cellular fluids, such as intracellular fluids and extracellular fluids. The resultant path for the emitted light from the optical emitter to the optical detector through biological tissues can assume a broad, arc-shaped optical path as it returns to the optical detector. The arc-shaped optical path becomes deeper as the distance between the emitter and detector increases (all other factors being equal).

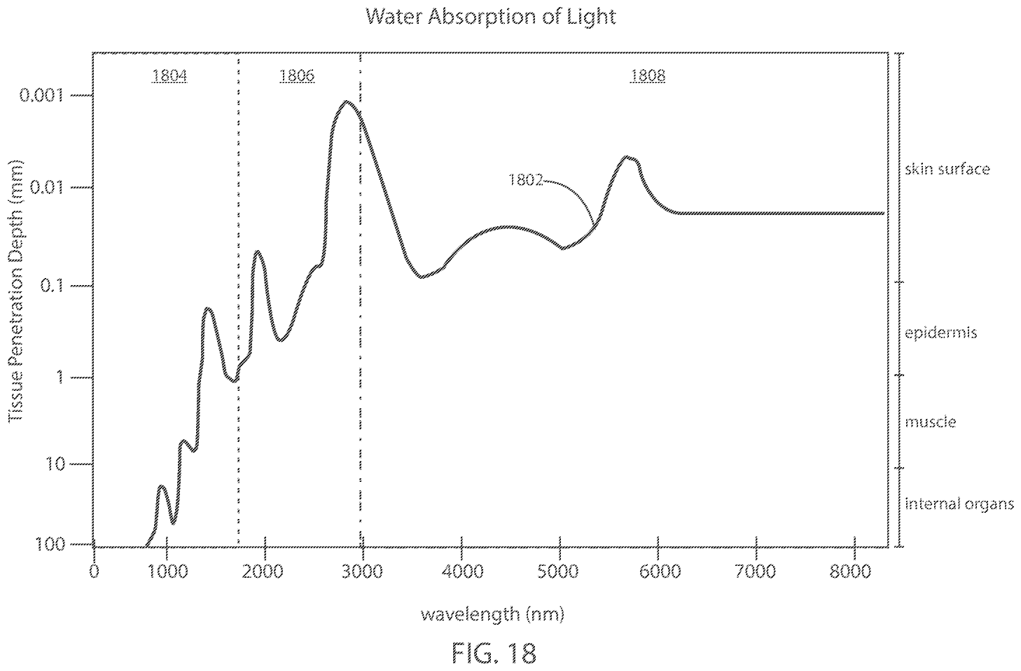

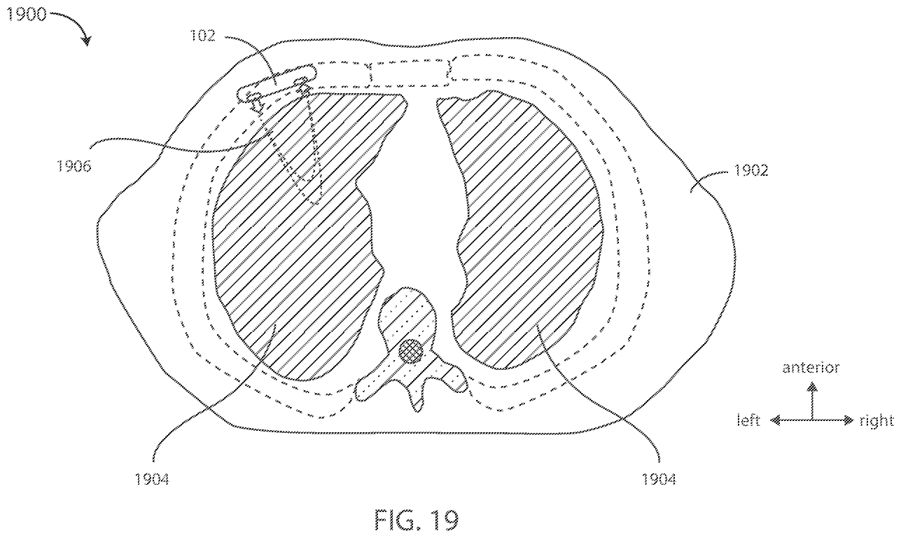

Referring now to FIG. 18, a graphical representation of water absorption of light at various wavelengths illustrating tissue penetration depth is shown in accordance with the embodiments herein. The tissue penetration depth in millimeters (mm) as a function of wavelength in nanometers (nm) is represented as plot 1802. At wavelengths of from 700 nm to 1500 nm, represented in section 1804 of plot 1802, the tissue penetration depth is from roughly 5 mm to 100 mm, which includes depths that penetrate within the epidermis, muscle, internal organs. At wavelengths of from 1500 nm to 3000 nm, represented in section 1806 of plot 1802, the tissue penetration depth is only from roughly 0.1 mm to 0.001 mm, which includes portions of the epidermis and the skin surface. At wavelengths greater than 3000 nm, represented in section 1808 of plot 1802, the tissue penetration depth is from 0.1 mm or less, which includes superficial portions of the epidermis and the skin surface. Thus, as can be seen, the specific wavelength chosen impacts the maximum depth of propagation through the tissue. In embodiments herein, wavelengths are selected (as also described elsewhere herein) to provide for deep tissue propagation of the emitted light.

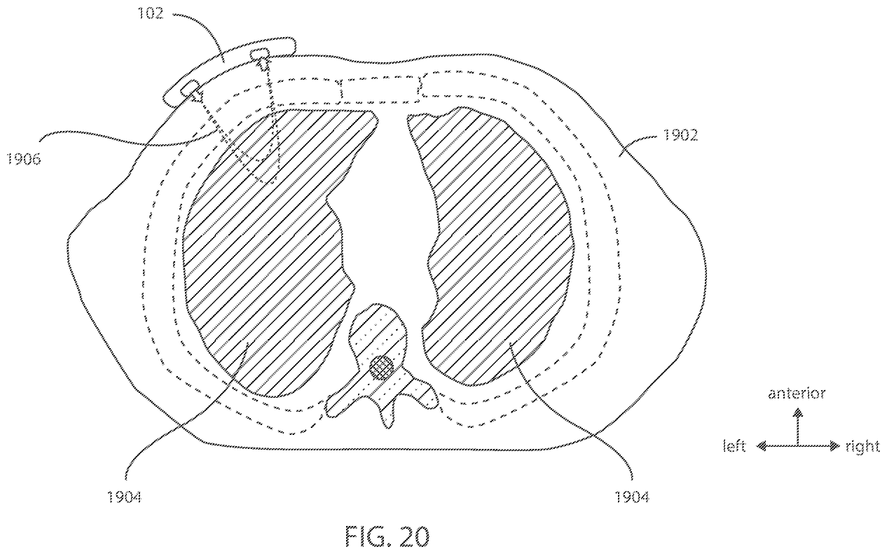

Referring now to FIG. 19, a schematic cross-sectional diagram of a human thorax 1902 with an implanted optical monitoring device positioned therein is shown in accordance with the embodiments herein. The cross-sectional view 1900 of the human thorax 1902 includes the left and right lungs 1904. An optical monitoring device 102 is shown positioned within an intercostal space with an optical emitter and an optical detector disposed facing a surface of the lung tissue. The optical monitoring device 102 is shown disposed facing the interior of a patient's body and is directed toward the surface of the tissue to be monitored. In some embodiments, the tissue to be monitored includes the airway, lung tissue, cardiovascular tissue, and the like. Implanting the optical monitoring device 102 facing the interior of a patient's body can selectively direct emitted light 1906 toward tissues to be illuminated inside the patient's body.

Referring now to FIG. 20, a schematic cross-sectional diagram of a human thorax 1902 with a wearable optical monitoring device positioned thereon is shown in accordance with the embodiments herein. The cross-sectional view 1900 of the human thorax 1902 includes the left and right lungs 1904. An optical monitoring device 102 having an optical emitter and optical detector is shown externally positioned on the skin over an intercostal space. The optical monitoring device 102 can be held in place using a wearable strap or another device. The optical monitoring device 102 is shown disposed facing the interior of a patient's body and is directed toward the surface of the tissue to be monitored. In some embodiments, the tissue to be monitored includes the airway tissue, lung tissue, cardiovascular tissue, or any other anatomical structure of interest in the body, and the like. Implanting the optical monitoring device 102 with the emitter(s) and detector(s) thereof facing the interior of a patient's body can selectively direct emitted light 1906 toward tissues to be illuminated inside the patient's body.

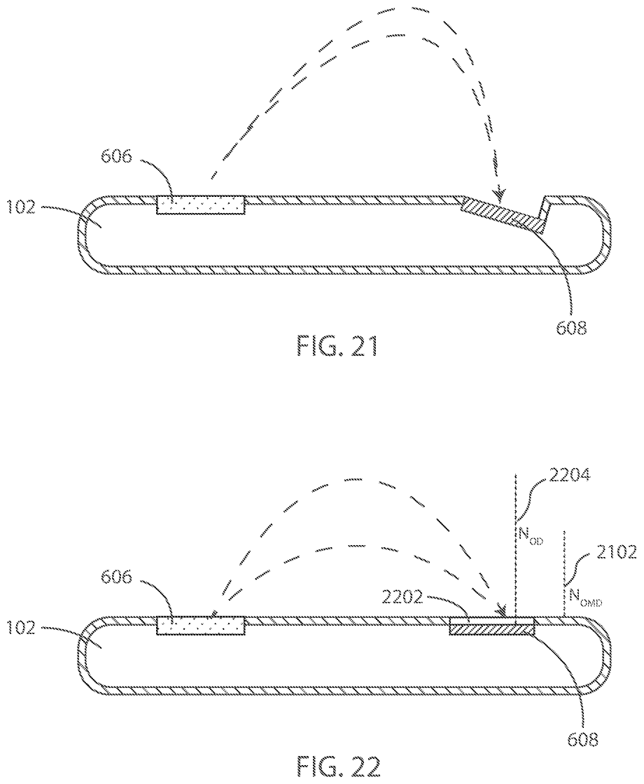

As discussed elsewhere herein, light can propagate through bodily tissues via random scattering as caused by the difference in the index of refraction of the various molecules and compounds in the tissues. Scattering generates light propagating through tissues at many angles, and the optical detectors described herein can be configured to selectively detect incident light with respect to its angle of incidence (or range of angles) on the optical monitoring device. In various embodiments, the first optical detector or a portion thereof can be angled with respect to a surface of the optical monitoring device. Referring now to FIG. 21, a schematic cross-sectional view of an optical monitoring device 102 is shown in accordance with various embodiments herein. In this embodiment, the optical monitoring device 102 includes a first optical emitter 606 and a first optical detector 608, each coupled to the housing as discussed elsewhere herein. The first optical detector 608 can be angled with respect to a surface normal (N_{OMD}) 2102 where N_{OMD} is the surface normal of the optical monitoring device. The angle of the first optical detector 608 relative to the surface normal (N_{OMD}) 2102 of the optical monitoring device 102 can include an angle of from −75 degrees to 75 degrees relative to the surface normal (N_{OMD}) 2102 of the optical monitoring device.

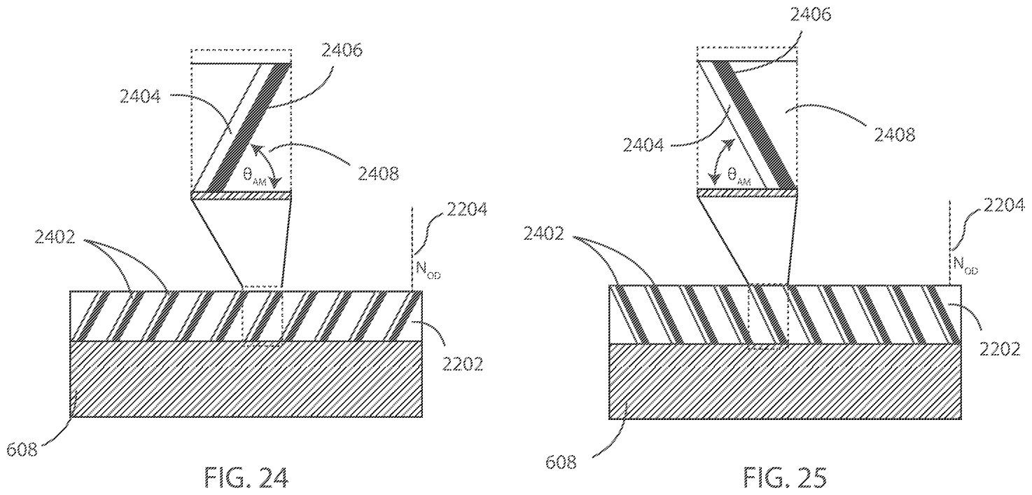

The optical detectors herein can further include an angularly selective filter disposed on a surface of the optical detectors. Referring now to FIG. 22, a schematic cross-sectional view of an optical monitoring device 102 is shown in accordance with various embodiments herein. In this embodiment, the optical monitoring device 102 includes a first optical emitter 606 and a first optical detector 608 having an angularly selective filter 2202 disposed on a surface thereof. The angularly selective filter can include a plurality of angled members, a grating optical filter, an alternating layer filter, a lens, a mirror, a collimator, and the like, as will be discussed in more detail with respect to FIGS. 24-26. The angularly selective filter 2202 can be configured to allow transmittance of light having various angles of incidence relative to the surface normal (N_{OD}) 2204 of the first optical detector 608 and further configured to block light having various angles of incidence relative to the surface normal (N_{OD}) 2204.

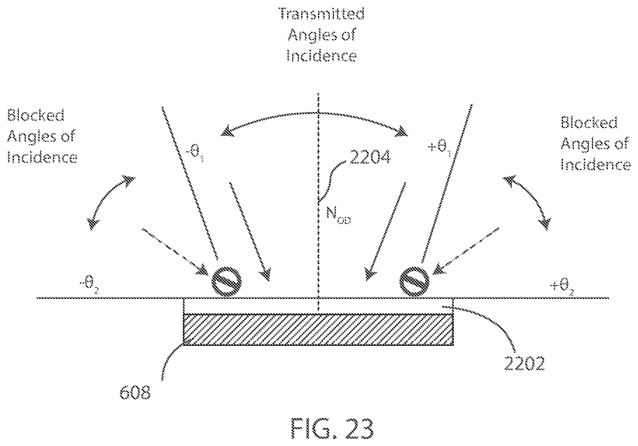

The angularly selective filters herein can be configured to selectively transmit incident light having a first incident angle or a first range of incident angles relative to the surface normal (N_{OD}) 2204 of the optical detectors and selectively block incident light having a second incident angle or second range (or ranges) of incident angles relative to the surface normal (N_{OD}) 2204 of the optical detectors from entering the detectors. Referring now to FIG. 23 a schematic diagram of incident light on an optical detector having an angularly selective filter 2202 disposed thereon is shown in accordance with various embodiments herein. The first optical detector 608 includes an angularly selective filter 2202 disposed at an exterior surface of the first optical detector 608. The angularly selective filter 2202 can be configured to allow for a single incident angle of light or a range of incident angles of light to be transmitted to the first optical detector 608. The transmitted angles of incident light can fall within a range of angles including from about −θ₁ to +θ₁ relative to a surface normal (N_{OD}) 2204 of the optical detector, where the range of −θ₁ to +θ₁ includes angles of from −75 degrees to +75 degrees.

In various embodiments, the value for the transmitted angles of incident light relative a surface normal (N_{OD}) can be greater than or equal to −75 degrees, −70 degrees, −65 degrees, −60 degrees, −55 degrees, −50 degrees, −45 degrees, −40 degrees, −35 degrees, −30 degrees, −25 degrees, −20 degrees, −15 degrees, −10 degrees, −5 degrees, 0 degrees, 5 degrees, 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, or 75 degrees, or can be an amount falling within a range between any of the foregoing. It should be understood the angle of the first optical detector can be relative to the short axis of optical monitoring device 102, the long axis of device 102 or both the short and long axis of device 102. In some embodiments, it will be appreciated that the value for the transmitted angles of incident light relative a surface normal (N_{OD}) can include those that run parallel to the surface normal (N_{OD}) of the optical detector.

The angularly selective filters herein can be configured to block incident light from the optical detectors at a single incident angle or a range of incident angles. In various embodiments, the range of incident angles of light that can be blocked by the angularly selective filters can include those having angles in a range of from +θ₂ to about −θ₁ or from about +θ₁ to +θ₂. In various embodiments, the value for the blocked angles of incident light relative a surface normal (N_{OD}) can be from −180 degrees, −170 degrees, −160 degrees, −150 degrees, −140 degrees, −130 degrees, −120 degrees, −110 degrees, −100 degrees, −90 degrees, or −80 degrees, or can be an amount falling within a range between any of the foregoing. In other embodiments, the value for the blocked angles of incident light relative a surface normal (N_{OD}) can be from 80 degrees, 90 degrees, 100 degrees, 110 degrees, 120 degrees, 130 degrees, 140 degrees, 150 degrees, 160 degrees, 170 degrees, or 180 degrees, or can be an amount falling within a range between any of the foregoing. In some embodiments, the angularly selective filters herein can be configured to block incident light from the optical detectors at two or more ranges of incident angles, where each range is independently selected from the ranges described above.

It will be appreciated that the angularly selective filters herein can be configured to simultaneously allow for transmission of light from within a range of incident angles including angles of from about −θ₁ to +θ₁ relative to a surface normal (N_{OD}) of the optical detector while blocking light having incident angles in a range of angles of from +θ₂ to about −θ₁ or from +θ₁ to about +θ₂.

In one approach, the angularly selective filters herein can include a plurality of angled members 2402 configured to allow for the selective transmission of incident light at a first incident angle or a first range of incident angles and selectively block incident light at a second incident angle(s) or second range(s) of incident angles. Referring now to FIGS. 24 and 25, a schematic cross-sectional view of an embodiment of an optical detector is shown in accordance with various embodiments herein. The first optical detector 608 includes an angularly selective filter 2202 disposed on a surface of the first optical detector 608. In various embodiments the angularly selective filter 2202 can be made from various materials to allow for the transmission of light including a light transmitting material 2404 made from a polymeric material, a glass, a ceramic, a mirror, and the like. In various embodiments, the angularly selective filter 2202 can further include various materials including light blocking materials such as opaque polymers, metals, dyed materials, and the like. It will be appreciated that in some embodiments, transmitting materials can include reflective materials such as mirrors, where the mirrors can include reflective properties and/or as transmissive properties.

Each of the individual angled members 2402 can include a light transmitting material 2404 and a light blocking material 2406 in various configurations. In the embodiment shown in FIG. 24, the light transmitting material 2404 is configured to be directed toward an exterior of the first optical detector 608 and the light blocking material 2406 is configured to be directed toward an interior of the first optical detector 608. In the embodiment shown in FIG. 25, the light transmitting material 2404 is configured to be directed toward an interior of the first optical detector 608 and the light blocking material 2406 is configured to be directed toward an exterior of the first optical detector 608. The angled members 2402 can be disposed within the angularly selective filter 2202 at an angle θ_{AM}, where AM stands for angled member, relative to a surface of the first optical detector 608.

The plurality of angled members 2402 can be embedded in a transparent material 2408 such as a polymeric layer or a glass layer. However, in some embodiments, the plurality of angled members are not embedded in a transparent material. Each of the individual angled members can be disposed within the angularly selective filter 2202 at an angle (θ_{AM}) of from 5 degrees to 75 degrees relative to a surface of the first optical detector 608. The angled members 2402 can be disposed in the angularly selective filter 2202 where the angle θ_{AM} can include an angle of from 5 degrees, 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, or 75 degrees.

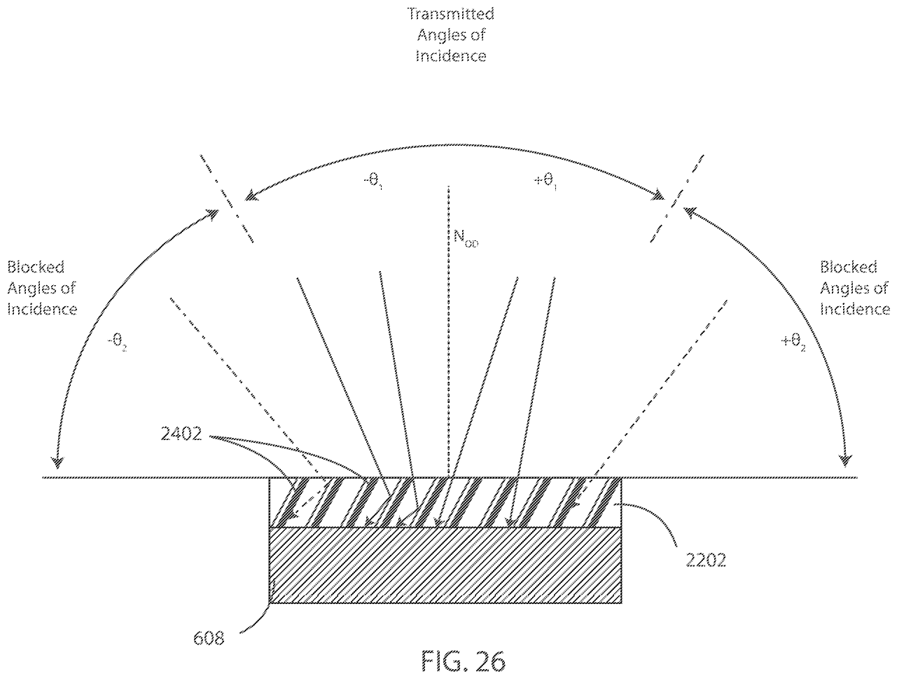

Referring now to FIG. 26, a schematic diagram of incident light on a first optical detector 608 having an angularly selective filter 2202 disposed thereon is shown in accordance with various embodiments herein. The first optical detector 608 includes an angularly selective filter 2202 having a plurality of angled members 2402 disposed at an exterior surface of the first optical detector 608. The angularly selective filter 2202 can be configured to allow for a single incident angle of light or a range of incident angles of light to be transmitted to the first optical detector 608. The transmitted angles of incident light can fall within a range of angles including from about −θ₁ to +θ₁ relative to a surface normal (N) of the optical detector, where the range of −θ₁ to +θ₁ includes angles of from −75 degrees to +75 degrees, as discussed in more detail with respect to FIG. 23.

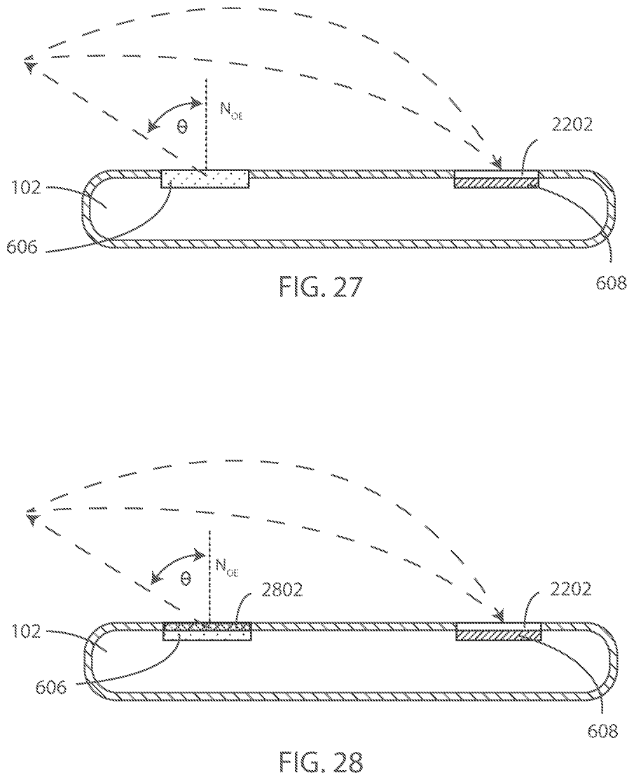

The optical monitoring devices herein can include optical emitters that are configured to emit light having a maximum intensity at a predetermined angle or range of angles, that in some embodiment can be in a direction along a vector that extends opposite the optical emitters. Referring now to FIG. 27, a schematic cross-sectional view of an embodiment of an optical monitoring device 102 in accordance with various embodiments herein. The optical monitoring device can include a first optical emitter 606 and a first optical detector 608. The first optical emitter 606 that can be configured to emit light in a direction away from the optical detector. In various embodiments, the optical emitter can be configured to emit light with a maximum intensity in a direction along a vector that extends at an angle θ of from 5 degrees to 75 degrees relative to a surface normal (N_{OE}) 2802 the first optical emitter.

Extending the length of the device can allow for a greater maximum separation between an emitter and detector, which can in turn increase the depth of light propagation through a tissue. However, the overall length of a device serves as a constraint on the maximum separation between an emitter and detector on the device and there are practical limits on how long a device can be. Without being bound by theory, it is believed that directional light emission (such as emitting light in a direction away from the optical detector) and/or directional light detection herein can achieve the same function as increasing the distance between the emitted light and optical detectors with respect to depth of light propagation. That is, the device and components thereof can be configured to provide for directional light emission (such as emitting light in a direction away from the optical detector) and/or directional light detection so that the light used by the device for measurement of physiological parameters and/or detection of conditions has propagated more deeply through the tissue. As such, the devices herein can be used to measure physiological parameters and/or detect conditions in tissue at depths greater than otherwise possible based on the overall length of the device.

Beyond enabling measurement of physiological parameters and/or detection of conditions more deeply within tissue, directional light emission and/or directional light detection herein can allow for the measurement of physiological parameters, and/or detection of conditions selectively at certain depths. For example, light that has propagated through tissue more deeply will generally come back to the detector with a maximum intensity at an angle of incidence that is different than light that has propagated through tissue more shallowly. Thus, embodiments herein with directional light emission and/or directional light detection herein can be configured to selectively utilize light having propagated through tissue at specific depths, or depth ranges, to measure physiological parameters and/or detect conditions at specific depths, or depth ranges.

As such, the optical emitters herein can be disposed along a length of the optical monitoring devices and oriented and/or configured with one or more filters configured to emit light with maximum intensity along a vector in a direction away from the optical detector. Referring now to FIG. 28, a schematic cross-sectional view of an embodiment of an optical monitoring device 102 is shown in accordance with various embodiments herein. In this embodiment, the first optical emitter 606 can include a directional emission filter 2802 disposed on a surface of the first optical emitter 606. In various embodiments, the directional emission filter 2802 can be configured to direct light emitted by the first optical emitter 606 with a maximum intensity in a direction along a vector that extends at an angle θ of from 5 degrees to 75 degrees relative to a surface normal (N_{OE}) 2802 the first optical emitter. The directional emission filter 2802 can include various materials and configurations. In various embodiments, the materials that create the direction emission filters can include, but are not limited to, mirrors, prisms, piezo actuators, diffraction gratings, an alternating layer filter, a lens, and a collimator.

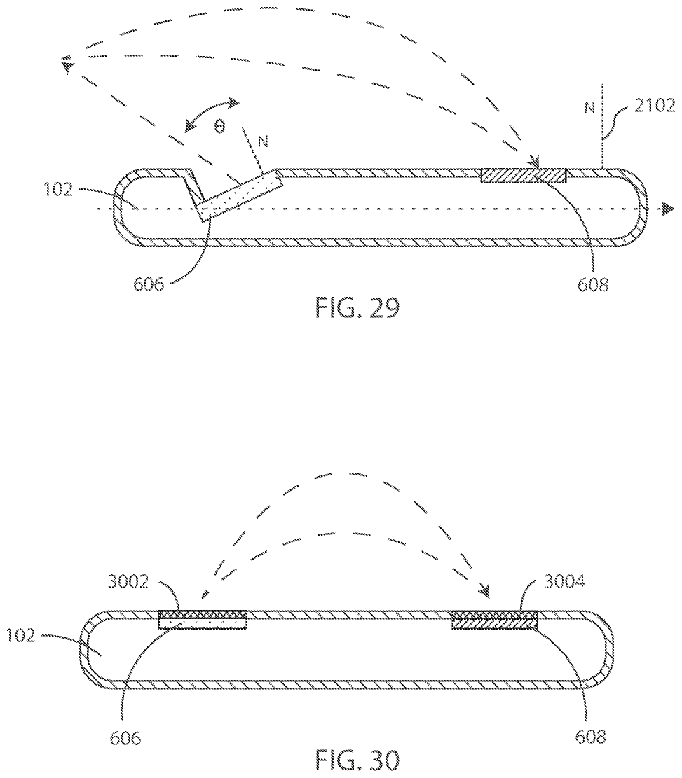

The optical emitters herein can be disposed along a length of the optical monitoring devices and configured (oriented or otherwise positioned) to be at an angle relative to a surface or longitudinal axis of the optical monitoring device 102 to emit light along a vector in a direction away from the first optical detector 608. Referring now to FIG. 29, a schematic cross-sectional view of an embodiment of an optical monitoring device is shown in accordance with various embodiments herein. In this embodiment, the optical monitoring device 102 includes a first optical emitter 606 and a first optical detector 608. The first optical emitter 606 can be disposed along the optical monitoring device 102 at an angle relative the longitudinal axis of the optical monitoring device 102. In various embodiments, the optical emitter can be configured to emit light with a maximum intensity in a direction along a vector that extends at an angle θ of from 5 degrees to 75 degrees relative to a surface normal the first optical emitter 606.

In various embodiments directional light emission and/or detection can allow for measurement of light that has propagated through specific tissues, for example light that has propagated through tissues to the right, left, forward, backward, above and/or below optical monitoring device 102. In an example light emission could be directed below and to the right of device 102 to selectively interrogate lung tissue which is not located in other directions.

In various embodiments directional light emission and/or propagation can be altered due to alterations in the orientation, location or angle of tissue relative to optical monitoring device 102. The alterations in directional light emission and/or propagation can be more-or-less periodic or aperiodic. In an embodiment the directional angle can be changed to compensate for periodic alterations of angle between the patient's lung tissue and device 102 due to the patient's respiration or heart beat. In another embodiment the directional angle can be changed to compensate for aperiodic alterations of angle between the patient's lung tissue and device 102 due to the patient's posture or to movement of device 102 within the body after implantation.

In various embodiments herein the optical emitters and optical detectors can include one or more layers of a polarizing material disposed on a surface thereof. Referring now to FIG. 30, a schematic cross-sectional view of an embodiment of an optical monitoring device is shown in accordance with various embodiments herein. The optical monitoring device 102 can include a first optical emitter 606 and a first optical detector 608. The first optical emitter 606 can be configured to emit light at a first wavelength as discussed elsewhere herein. The first optical detector 608 can be configured to detect incident light. The first optical emitter 606 can include a first polarization material 3002 disposed on at least a portion of a surface of the first optical emitter 606. The first optical detector 608 can include a second polarization material 3004 disposed on at least a portion of a surface of the first optical detector 608. In various embodiments, the first polarization material 3002 is disposed on the entire surface of the first optical emitter 606 and the second polarization material 3004 can be disposed on the entire surface of the first optical detector 608. In various embodiments, a first polarization material 3002 can be disposed on at least a portion of the first optical emitter 606 while no polarization material is disposed on the first optical detector 608. In some embodiments, a second polarization material 3004 can be disposed on at least a portion of the first optical detector 608, while not polarization material is disposed on the first optical emitter 606. In various embodiments, the first polarization material 3002 can be disposed about a perimeter of the first optical emitter 606 and the second polarization material 3004 can be disposed about a perimeter of the first optical detector 608.

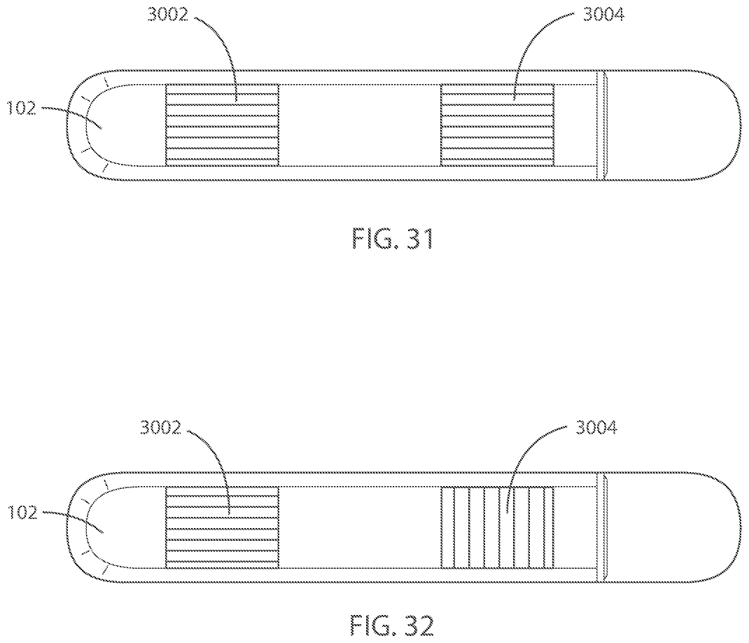

The first polarization materials and second polarization materials can be disposed on the surfaces of the first optical emitter and first optical detector in various orientations. In various embodiments, the polarization axis of the first polarization material can be oriented parallel to a polarization axis of the second polarization material. Referring now to FIG. 31, a schematic top view of an embodiment of an optical monitoring device 102 is shown in accordance with various embodiments herein. The optical monitoring device 102 includes a first polarization material 3002 can be oriented parallel to a polarization axis of the second polarization material 3004. In various embodiments, the polarization axis of the first polarization material can be oriented perpendicular to a polarization axis of the second polarization material. Referring now to FIG. 32 is a schematic top view of an embodiment of an optical monitoring device 102 in accordance with various embodiments herein. The optical monitoring device 102 includes a first polarization material 3002 can be oriented perpendicular to a polarization axis of the second polarization material 3004.

The polarization axis of the first polarization material can be oriented at an angle in a range of from 0 degrees to 90 degrees to a polarization axis of the second polarization material. In some embodiments, the first polarization material can be oriented at an angle that can be greater than or equal to 0 degrees, 5 degrees, 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 85 degrees, or 90 degrees relative to the polarization axis of the second polarization material, or can be an amount falling within a range between any of the foregoing.

The polarization materials can be made from various materials, including but not to be limited to one or more of polymers, glasses, ceramics, dichroic absorption layers, and the like. In various embodiments, the first polarization material and the second polarization material can each comprise a diffraction grating. In various embodiments, the first polarization material and second polarization material are the same, while in some embodiments the first polarization material and second polarization material are different.

### Emitter/Detector Characteristics

In some embodiments, the one or more optical emitters can include solid state light sources such as GaAs, GaAlAs, GaAlAsP, GaAlP, GaAsP, GaP, GaN, InGaAlP, InGaN, ZnSe, or SiC light emitting diodes or laser diodes that excite the sensing one or more optical detectors at or near the wavelength of maximum absorption for a time sufficient to emit a return signal. However, it will be understood that in some embodiments the wavelength of maximum absorption/reflection varies as a function of the optical path from the one or more optical emitters to the one or more optical detectors.

In some embodiments, the one or more optical emitters can include other light emitting components including incandescent components. In some embodiments, the optical emitters can include a waveguide. The optical emitters can also include one or more filters such as bandpass filters, high pass filter, low pass filters, and/or other components such as antireflection elements, and/or focusing optics.

In some embodiments, the one or more optical emitters can include a plurality of LEDs with bandpass filters, with each of the LED-filter combinations emitting at a different center frequency. According to various embodiments, the LEDs can operate at different center-frequencies, sequentially turning on and off during a measurement. As multiple different center-frequency measurements are made sequentially, a single unfiltered optical detector can be used in some embodiments. However, in some embodiments, a polychromatic source can be used with multiple detectors that are each bandpass filtered to a particular center frequency.

The one or more optical detectors can be configured to receive light from the optical emitters. In an embodiment, the optical detectors can include a component to receive light. By way of example, in some embodiments, the optical detectors can include a charge-coupled device (CCD). In other embodiments, the optical detectors can include a photodiode, a junction field effect transistor (JFET) type optical sensor, or a complementary metal-oxide semiconductor (CMOS) type optical sensor. In some embodiments, the optical detectors can include an array of optical detecting components. In some embodiments, the optical detectors can include a waveguide. The one or more optical detectors can also include one or more bandpass filters and/or focusing optics. In some embodiments, the optical detectors can include one or more photodiode detectors, each with an optical bandpass filter tuned to a specific wavelength range.

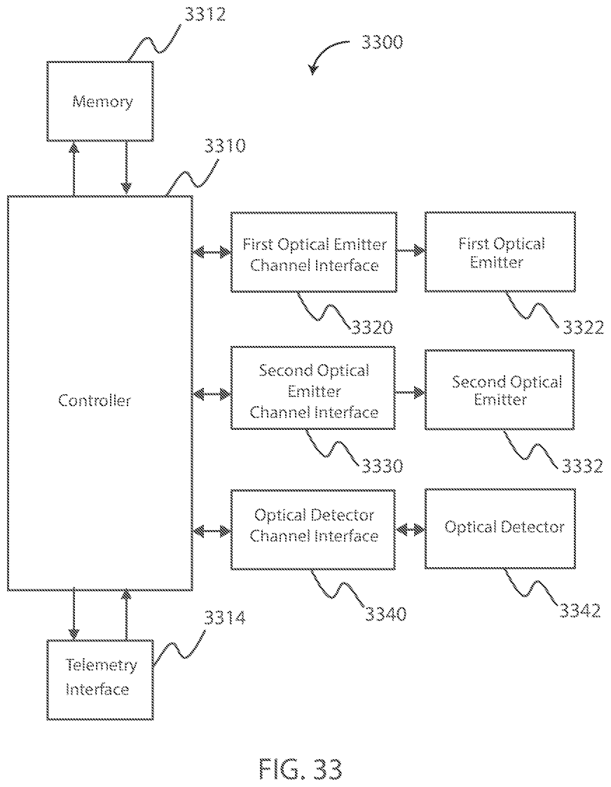

Referring now to FIG. 33, a schematic block diagram of some components 3300 of an optical monitoring device is shown in accordance with various embodiments herein. It will be appreciated that some embodiments can include additional elements beyond those shown in FIG. 33. In addition, some embodiments may lack some elements shown in FIG. 33. The implantable sensing devices herein can gather information through one or more detecting channels. A controller 3310 can communicate with a memory 3312 via a bidirectional data bus. It will be appreciated that controller 3310 can include one or more microprocessors. The memory 3312 can include read-only memory (ROM) or random-access memory (RAM) for program storage and RAM for data storage, or any combination thereof. The implantable medical device can include one or more optical emitters 3322, 3332, one or more detectors 3342, or one or more other sensors (not pictured). The one or more other sensors, but are not to be limited to, a pulse oximetry sensor, a chemical sensor, a posture sensor, or a heart rate sensor. The optical monitoring device can further include a measurement circuit configured to control operation of the optical emitters and the optical detectors.

Each optical emitter 3322, 3332 is communicatively coupled to a light emitter channel interface 3320, 3330. Each detector 3342 is communicatively coupled to a detector channel interface 3340. Each other sensor is communicatively coupled to a separate and other sensor channel interface (not pictured). Each of the light emitter channel interfaces 3320, 3330, the detector channel interface 3340, and any of the other sensor channel interfaces can communicate with controller 3310.

The first light emitter channel interface 3320, the second light emitter channel interface 3330, and the detector channel interface 3340 can each include various components such as analog-to-digital converters for digitizing signal inputs, sensing amplifiers, registers which can be written to by the control circuitry in order to adjust the gain and threshold values for the sensing amplifiers, source drivers, modulators, demodulators, multiplexers, and the like. A telemetry interface 3314 is also provided for communicating with external devices such as a programmer, a home-based unit, and/or a mobile unit (e.g., a cellular phone, portable computer, etc.), implanted devices such as a pacemaker, cardioverter-defibrillator, loop recorder, and the like.

### AC/DC Component Calculations

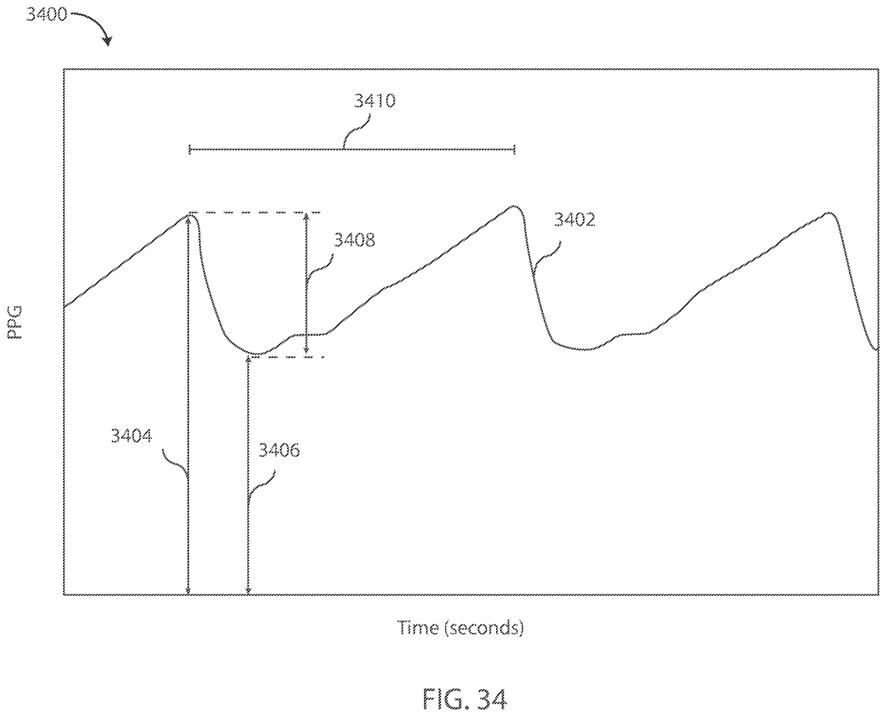

Referring now to FIG. 34, a graphical representation of an optical signal measured by an optical monitoring device as described herein is shown in accordance with the embodiments herein. The optical signal 3400 can be indicative of the incident light measured by the optical detector of an optical temperature measurement device. As depicted by FIG. 34, the optical signal can be approximately periodic having period 3410, maximum value 3404, and minimum value 3406. In this example, the periodicity of the signal is a consequence of the systole and diastole of the cardiac cycle. During systole, the heart contracts, pushing the oxygenated blood towards the periphery of the body, resulting in an increased blood pressure. During diastole, the heart fills with blood, retracting the blood from the periphery of the body resulting in a decrease in blood pressure. Due to the absorption of light by various chromophores in blood, such as oxyhemoglobin and deoxyhemoglobin, the optical density of the tissue at certain wavelengths can increase during systole and decreases during diastole resulting, in a periodic signal with maximums and minimums. Each cycle of the signal contains a maximum value 3404 at the systolic peak and a minimum value 3406 at the diastolic valley.

The optical signal can effectively have an AC (or variable) component 3408 and a DC (or constant) component 3402. The term ‘AC’, as used herein, refers to a portion of signal that varies relatively rapidly with time. The AC component can be indicative of the portion of the signal originating by pulsations in a patient's blood during each heartbeat. The term ‘DC’, as used herein signals, refers to portions of the signal that are relatively invariant with time.

In various embodiments, both an AC component and a DC component of the light detected by the optical detector can be used to determine a parameter of the tissue (e.g., temperature, oxygenation, congestion, cardiac oscillations). In various embodiments, the parameter can be derived from a ratio of the AC and DC components of the signal as measured by the optical detector. In various embodiments, the parameter can be derived from a ratio of the AC and DC components of the signal measured by the optical detector at two distinct wavelengths. For instance, the property can be derived using a ratio of ratios (RoR) calculation according to the following calculation:

$$temperature ∝ \frac{{AC}_{1} / {DC}_{1}}{{AC}_{2} / {DC}_{2}}$$

where AC₁ is the AC component of the signal measured by the optical detector at the first wavelength; DC₁ is the DC component of the signal measured by the optical detector at the first wavelength; AC₂ is the AC component of the signal measured by the optical detector at the second wavelength; and DC₂ is the DC component of the signal measured by the optical detector at the second wavelength.

### Tissue Penetration

Spacing of the optical emitters and optical detectors along a length of the optical monitoring devices herein can impact the depth of penetration of the emitted light into the surrounding tissues. By way of non-limiting example, the penetration depth of emitted light can be approximated according to the following equation (without accounting for the impact of directional light emission and/or directional light detection as described herein):

light penetration depth=˜1/2(spacing between emitter and detector).

In some embodiments, the depth of propagation of light from the emitter light can be greater than or equal to 1 cm, 2 cm, 3 cm, 4 cm, or 5 cm, or can be an amount falling within a range between any of the foregoing. In various embodiments, the configuration of the optical monitoring device can allow for the propagation of the emitted light into a patient's body and into the tissue thereof. In some embodiments, the length between the first optical emitter and the first optical detector can be greater than or equal to 1 cm, 2 cm, 3 cm, 4 cm, 5 cm, 6 cm, 7 cm, 8 cm, 9 cm, 10 cm, 11 cm, 12 cm, 13 cm, 14 cm, or 15 cm, or can be an amount falling within a range between any of the foregoing.

However, due to the impact of directional light emission and/or directional light detection as described herein, the depth of propagation of light through tissues as used to measure physiological parameters and/or detect conditions can be deeper than would otherwise be possible based on spacing of optical emitters and optical detectors. In various embodiments herein, the depth of propagation of light through tissues as used to measure physiological parameters and/or detect conditions can be at least 0.5, 1, 2, 3, 4, or 5 cm or more deeper than would otherwise be the case if only the spacing between optical emitters and optical detectors was relied upon.

### Therapies

The devices and systems herein can be configured to suggest, initiate and/or deliver a therapy to a patient in response to a change in a physiological parameter detected by the optical monitoring devices herein. In various embodiments, the optical monitoring devices can be configured to deliver a therapy to a patient. In some embodiments, implantable therapeutic devices can be implanted and used in a system in conjunction with the wearable optical monitoring devices described herein. The implantable therapeutic devices can include those that deliver an electrical stimulation therapy as well as other types of therapy. The implantable therapeutic devices can include, but not be limited to cardiac rhythm management devices, an implantable cardioverter-defibrillator (ICD), a left ventricular assist device (LVAD), a pacemaker, and the like.

In various embodiments, the devices and systems herein can be configured to deliver or modify a pulmonary oxygenation therapy to a patient to treat one or more diseases states or conditions including, but not limited to, chronic obstructive pulmonary disease (COPD), pulmonary edema with heart failure with normal cardiac output or reduced cardiac output, asthma, pneumonia, various airway obstructions, and pulmonary embolism. In some embodiments, a pulmonary oxygenation therapy can include a command sent to a device such as a ventilation device or other breathing assistance device to change proportions of gases being provided (such as increasing or decreasing oxygen content), change volumes of gases being provided, or the like.

### Methods

Many different methods are contemplated herein, including, but not limited to, methods of making, methods of using, and the like. Aspects of system/device operation described elsewhere herein can be performed as operations of one or more methods in accordance with various embodiments herein.

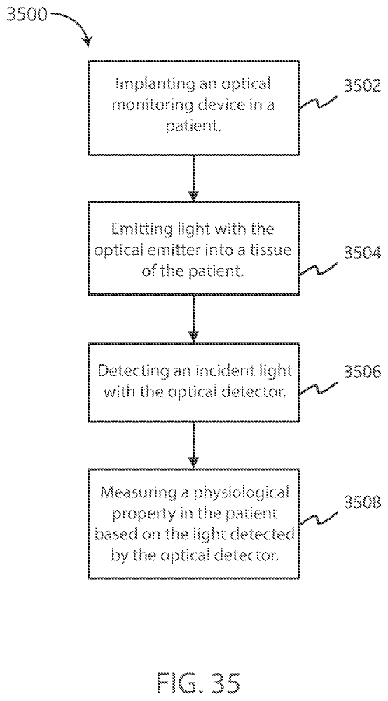

Referring now to FIG. 35, a method 3500 for measuring and determining a physiological parameter in a patient is included. The method can include implanting an optical monitoring device in a patient at 3502 where the optical monitoring device can include an optical emitter and an optical detector. The method can include emitting light with the optical emitter into a tissue of the patient at 3504. The method can include detecting incident light with the optical detector at 3506. The method can include measuring a physiological parameter and/or detecting a condition in the patient based on the light detected by the optical detector at 3508.

In an embodiment of the method, implanting the optical monitoring device comprises implanting the optical monitoring device subcutaneously at or near a site of lung tissue or other tissue.

In an embodiment of the method, implanting the optical monitoring device subcutaneously comprises implanting the optical monitoring device in an intercostal space at or near the site of lung tissue or other tissue.

In an embodiment of the method, the optical monitoring device is implanted at a distance of from 5 mm to 50 mm from a surface of lung tissue or other tissue.

In an embodiment of the method, emitting light with the optical emitter into the lung tissue of the patient comprises emitting the light at a wavelength from 100 nm to 2000 nm.

In an embodiment of the method, emitting light with the optical emitter into the lung tissue of the patient comprises emitting the light at at least two distinct wavelengths.

In an embodiment of the method, implanting an optical monitoring device in a patient comprises orienting the optical monitoring device such that optical emitter and the optical detector face internally relative to the skin of the patient.

In an embodiment of the method, emitting light with the optical emitter into the lung tissue of the patient comprises propagating the light from 1 cm to 5 cm into the tissue of the patient relative to a surface of the optical monitoring device.

### Systems

Systems herein can include one or more optical monitoring devices and one or more secondary sensors. Aspects of optical monitoring devices described elsewhere herein can be included with one or more embodiments of a system in accordance with various embodiments herein.

In an embodiment, an optical monitoring system is included for detecting a physiological parameter of a patient. In various embodiments, the physiological parameter can include one or more of an oxygenation status of lung tissue, a pulmonary congestion status of lung tissue, a temperature, or a cardiogenetic airway modulation status. The optical monitoring system can include a housing. The optical monitoring system can include an optical monitoring device. The optical monitoring device can include an optical emission assembly. The optical emission assembly can include a first optical emitter configured to emit light at a first wavelength and a second optical emitter configured to emit light at a second wavelength. The optical monitoring device can include an optical detector configured to detect incident light. In the systems described herein the light emitted from the optical emitter propagates through lung tissue. In the systems described herein, the detected incident light is used to measure a physiological parameter of a patient. In the systems described herein, the optical monitoring device is configured to be implanted in a subcutaneous tissue of a patient.

In an embodiment of the system at least one of absorption, scattering and phase of the light detected by the optical detector is used to measure the physiological parameter.

In an embodiment of the system, both an AC component and an DC component of the light detected by the optical detector are used to measure the physiological parameter of a patient.

In an embodiment of the system, the first wavelength is from 100 nm to 2000 nm. In an embodiment of the system, the first wavelength is from 150 nm to 250 nm.

In an embodiment of the system, the second wavelength is a near-infrared wavelength. In an embodiment of the system, the second wavelength is from 750 nm to 1500 nm.

In an embodiment of the system, the physiological parameter of a patient can be a function of the ratio of the detected light originating from the first optical emitter and the detected light originating from the second optical emitter.

In an embodiment of the system, the housing comprising a flexible member extending from the housing, wherein at least one of the first optical emitter, the second optical emitter, and the optical detector are disposed on a flexible member.

In an embodiment of the system, the optical emission assembly and the optical detector are disposed on a substantially planar surface.

In an embodiment of the system, the optical emitter is disposed from 1 cm to 10 cm away from the optical detector.

In an embodiment of the system, the optical monitoring device is further configured to determine one of a heart rate, a respiratory rate, a tidal volume, or an extravascular lung water concentration.

In an embodiment of the system, the light from the optical emission assembly propagates a depth of 1 cm to 5 cm into the lung tissue as measured from a surface of the optical monitoring device.

In an embodiment of the system, the system further includes at least one of a secondary sensing including a pulse oximetry sensor, chemical sensor, posture sensor, and heart rate sensor.

In an embodiment of the system, the physiological parameter measurements are interpreted in view of at least one of heart rate, respiration, circadian rhythm, and posture.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

The claims are:

1. An optical monitoring device comprising:
a first optical emitter, wherein the first optical emitter is configured to emit light at a first wavelength; and
a first optical detector, wherein the first optical detector is configured to selectively detect incident light with respect to its angle of incidence on the optical monitoring device;
wherein the first optical emitter is configured so that the emitted light from the optical emitter propagates through a tissue at a depth of at least 1 cm into the tissue as measured from a surface of the optical monitoring device; and
wherein the optical monitoring device is configured to determine a physiological parameter of the tissue using incident light detected by the first optical detector.

2. The optical monitoring device of claim 1, wherein the first optical detector or a portion thereof is angled with respect to a surface of the optical monitoring device.

3. The optical monitoring device of claim 1, wherein the first optical detector comprises an angularly selective filter.

4. The optical monitoring device of claim 3, wherein the angularly selective filter comprises a plurality of angled members, a grating optical filter, an alternating layer filter, a lens, a mirror, or a collimator.

5. The optical monitoring device of claim 3, wherein the angularly selective filter is configured to selectively transmit incident light at a first incident angle or a first range of incident angles and selectively block incident light at a second incident angle or second range of incident angles.

6. The optical monitoring device of claim 4, wherein the plurality of angled members comprise an angle of from 5 degrees to 75 degrees relative to a surface normal of the angularly selective filter.

7. The optical monitoring device of claim 1, wherein the first optical detector is configured to detect incident light having a first incident angle of from −75 degrees to +75 degrees relative to the surface normal.

8. The optical monitoring device of claim 1, wherein propagation of emitted light through a lung tissue comprises propagation of emitted light from 1 cm to 5 cm in depth as measured from a surface of the optical monitoring device.

9. The optical monitoring device of claim 1, wherein the first optical emitter and the first optical detector are spaced along a planar surface of the optical monitoring device of from 1 cm to 10 cm apart.

10. An optical monitoring device comprising:
a first optical emitter, wherein the first optical emitter is configured to emit light with a maximum intensity at a predetermined angle from the first optical emitter; and
a first optical detector, wherein the first optical detector is configured to detect incident light;
wherein the light from the optical emitter propagates through a tissue;
wherein the incident light detected by the first optical detector is used to determine a physiological parameter of the tissue; and
wherein the light from the optical emitter propagates at a depth of at least 1 cm into the tissue as measured from a surface of the optical monitoring device.

11. The optical monitoring device of claim 10, wherein the first optical emitter is configured to emit light at a first wavelength.

12. The optical monitoring device of claim 10, wherein the first optical emitter and the first optical detector are spaced along a planar surface of the optical monitoring device of from 1 cm to 10 cm apart.

13. The optical monitoring device of claim 10, wherein the first optical emitter is configured to emit light with a maximum intensity in a direction along a vector that extends at an angle of from 5 degrees to 75 degrees relative to a surface normal the first optical emitter.

14. The optical monitoring device of claim 10, wherein the first optical emitter is disposed along the optical monitoring device at an angle relative the longitudinal axis of the optical monitoring device.

15. An optical monitoring device comprising:
a first optical emitter, wherein the first optical emitter is configured to emit light at a first wavelength;
a first optical detector, wherein the first optical detector is configured to detect incident light;
a first polarization material disposed on at least a portion of a surface of the first optical emitter; and
a second polarization material disposed on at least a portion of a surface of the first optical detector;
wherein the light from the optical emitter propagates through a tissue;
wherein the incident light detected by the first optical detector is used to determine a physiological parameter of the tissue; and
wherein the light from the optical emitter propagates at a depth of at least 1 cm into the tissue as measured from a surface of the optical monitoring device.

16. The optical monitoring device of claim 15, wherein the first polarization material is disposed on an entire surface of the first optical emitter and the second polarization material is disposed on an entire surface first optical detector.

17. The optical monitoring device of claim 15, wherein a polarization axis of the first polarization material is oriented parallel to a polarization axis of the second polarization material.

18. The optical monitoring device of claim 15, wherein a polarization axis of the first polarization material is oriented perpendicular to a polarization axis of the second polarization material.

19. The optical monitoring device of claim 15, wherein a polarization axis of the first polarization material is oriented at an angle in a range of from 0 degrees to 90 degrees to a polarization axis of the second polarization material.

20. The optical monitoring device of claim 15, wherein the first polarization material is disposed about a perimeter of the first optical emitter and the second polarization material is disposed about a perimeter of the first optical detector.
